(19) United States
(12) Patent Application Publication
(10) Pub. No.: US 2023/0374174 A1
(43) Pub. Date: Nov. 23, 2023

(54) Circularly Recyclable Polymers Featuring Topochemically Elongated Carbon-Carbon Bonds

(71) Applicant: Purdue Research Foundation, West Lafayette, IN (US)

(72) Inventors: Letian Dou, Zionsville, IN (US); Zitang Wei, West Lafayette, IN (US); Qixuan Hu, West Lafayette, IN (US); Xuyi Luo, Lafayette, IN (US)

(73) Assignee: Purdue Research Foundation, West Lafayette, IN (US)

(21) Appl. No.: 18/123,018

(22) Filed: Mar. 17, 2023

Related U.S. Application Data
(60) Provisional application No. 63/320,894, filed on Mar. 17, 2022.

(51) Int. Cl. C08F 136/14; C08F 132/08; C08F 132/06; C08J 11/12; C07C 67/293; C07C 253/30; C07C 67/333

(52) U.S. Cl. CPC C08F 136/14; C08F 132/08; C08F 132/06; C08J 11/12; C07C 67/293; C07C 253/30; C07C 67/333; C08J 2345/00; C08J 2347/00; C07C 2602/08

(57) ABSTRACT

A topochemical approach for creating elongated C—C bonds with a bond length of 1.57˜1.70 Å between monomers in the solid state; and topochemically prepared highly crystalline polymers.

### CROSS-REFERENCE TO RELATED APPLICATIONS

This application claims the benefit of Provisional U.S. Patent Application No. 63/320,894, filed Mar. 17, 2022, the disclosure of which is incorporated herein by reference in its entirety.

### BACKGROUND

### Technical Field

The current disclosure relates to the preparation of crystalline polymers with elongated carbon-carbon singles bonds that are suitable for a method of depolymerization at reduced temperatures.

### Description of Related Art

Increasing use of plastics causes serious environmental pollution and is a source of significant petroleum consumption. To date, there is no clear solution for a closed-loop circular utilization of polymers and plastics. From a fundamental chemistry standpoint, the bonds connecting the monomers in a conventional polymer are strong and not easily breakable. Recently, efforts have been made in the design of novel degradable polymers based on dynamic and cleavable C—O (ester), C—S(thioester), and C═N (enamine) bonds, and useful properties have been demonstrated. Creating controllable dynamic and reversible C—C bonds in a polymer using conventional synthetic approaches remains challenging because of their inherently large bond strength, high ceiling temperature (at which polymerization-depolymerization reaches equilibrium), and lack of effective catalysts. As a result, depolymerization/degradation normally happens at high temperatures along with a variety of side reactions in conventional polymers. A fully chemically recyclable hydrocarbon polymer with low-energy footprint remains elusive. Addressing this fundamental question is crucial because more than 90% of plastics produced today are hydrocarbon derivatives with monomer units bonded by simple C—C bonds.

A possible means of decreasing the strength of the C—C bond, while maintaining the levels required for function, is to elongate controllably the bond and decrease the sp³-orbital overlap between the two carbon atoms. In fact, elongated C—C bonds are well-understood in small molecules, and a few surprisingly long bonds have been reported. For example, a hexaphenylethane derivative developed by Mislow et al. exhibits a weak C—C bond in the molecule with a bond length of 1.68 Å. Later, more molecular systems were explored, including the diamondoid by Schreiner et al. and polycyclic hydrocarbon by Suzuki et al., with C—C bond lengths beyond 1.7 Å. Very recently, an impressive extra-long C—C bond length of 2.04 Å was achieved by Kubo et al. also using a polycyclic hydrocarbon strategy. Given the fact that C—C bond lengths have a quasi-linear relationship to the bond dissociation energies (BDE), as evidenced by both experimental and computational results of a large number of examples, it is expected that decreasing the strength of the C—C bond between the repeating units in a polymer can also be achieved by controllably elongating the C—C bond. Nevertheless, C—C bond elongation has not been well understood in polymers. In common semi-crystalline hydrocarbon-based polymers, such as polyethene (PE), polystyrene (PS) and polymethylmethacrylate (PMMA), all C—C bonds exhibit standard lengths of 1.53-1.54 Å between the repeating units. Creating an elongated C—C bond in polymers using conventional polymer synthesis strategies is challenging, because the necessary steric hindrance around the carbon atom will dramatically decrease the polymerization reactivity. Alternatively, we speculate that this circumstance can be perfectly avoided in solid-state topochemical polymerization, where monomer molecules have been pre-set at the right sites and are positioned to react. In this way, adding extra steric hindrance to fine-tune the bond length is possible. Furthermore, topochemical polymerizations are known to produce high quality stereoregular and ultra-high-molecular-weight crystalline polymers. Thus, this strategy would potentially provide polymer systems that perform similarly to traditional crystalline polymers while easily depolymerize to monomers through thermal cleavage of the relatively weak C—C bond.

### BRIEF SUMMARY

The present disclosure relates to elongated carbon-carbon bonds for fully depolymerizable polyolefin-derived materials. In one embodiment, the disclosure is directed to a crystalline polymer comprising two monomers wherein the monomers are chemically bound by at least one carbon-carbon single bond to form a crystalline polymer, wherein the carbon-carbon single bond has a bond length between about 1.57 Å and about 1.70 Å, and the polymer has a depolymerization temperature between about 125° C. to about 375° C. In another embodiment, the disclosure is directed to crystalline polymers derived from 1, 2 or 1, 4 polymerization. In one embodiment, the disclosure is directed to producing a polymer by mixing a crystalline polymer with a solvent, sonicating, vacuum filtering and heat pressing. In one embodiment, a thermoplastic is produced by mixing a crystalline polymer with a solvent, heating, mixing with a second solvent, and subsequently drying. In one embodiment, a method of depolymerization involves mixing the crystalline polymer with a solvent, heating to a specific temperature range and recovering the monomers.

A crystalline polymer comprising:
at least two monomers, which can be the same or different,
the at least two monomers are chemically bound by at least one carbon-carbon single bond to form the crystalline polymer,
wherein the carbon-carbon single bond has a bond length between about 1.57 Å and about 1.70 Å, and
wherein the polymer has a depolymerization temperature between about 125° C. to about 375° C. The bond length can be between about 1.59 Å and about 1.67 Å or between about 1.61 Å and about 1.63 Å. The depolymerization temperature can be between about 175° C. and about 220° C., between about 240° C. and about 275° C., or between about 310° C. and about 355° C.

A crystalline polymer comprising:
at least two monomers, independently selected from the group consisting of Formula I, Formula II, and Formula III,
wherein Formula I is

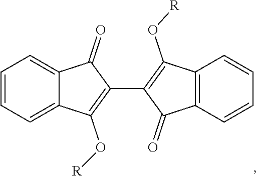

wherein each R is independently a C₁ to C₂₀ linear chain alkyl or a C₁ to C₂₀ branched chain alkyl, in which any —H may be substituted with —F,
wherein Formula II is

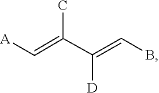

wherein A, B, C and D are independently selected from —C(═O)—R, —C(═O)—OR

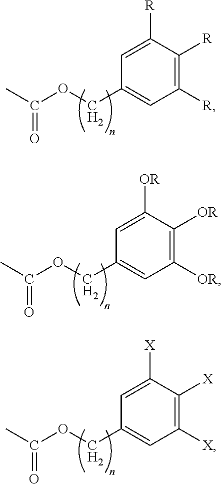

C(═S)—R,

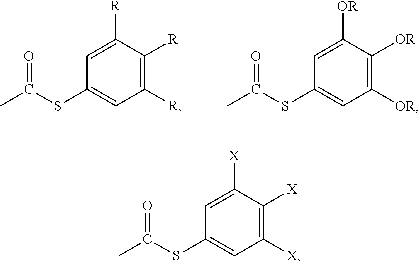

—C(═O)—NRR,

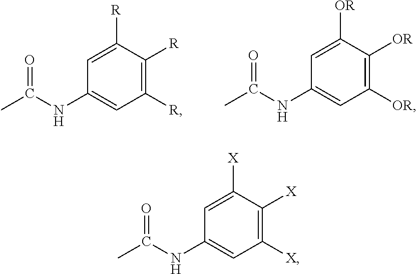

—C(═O)OM, —S(═O)OM, —S(═O)(═O)OM,

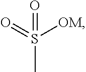

—NO₂, —OR, —OC(═O)R,

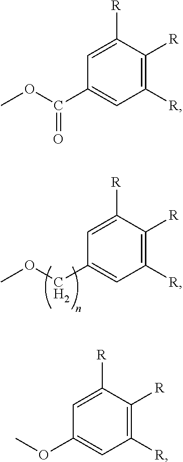

—H, —F, —Cl, and —CH₃,
wherein each R is independently H, —NO₂, a C₁ to C₂₀ branched chain alkyl, or a C₁ to C₂₀ straight chain alkyl, wherein the branched chain alkyl or the straight chain alkyl is optionally substituted with —O—, —NRR or —S—, and any hydrogen can be substituted with —F,
wherein X is a halogen or hydrogen, at least one X is a halogen, M is an organic or inorganic cation, and n=0 to 20 and
wherein Formula III is

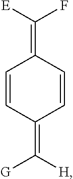

wherein each E, F, G, and H is independently —C(C═O)OR or CN, wherein each R is independently a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein the monomers are chemically bound to each other by at least one carbon-carbon single bond to form the crystalline polymer. The carbon-carbon bond can have a bond length between about 1.57 Å and about 1.70 Å, between about 1.59 Å and about 1.67 Å or between about 1.61 Å and about 1.63 Å. The polymer can have a depolymerization temperature between about 125° C. to about 375° C., between about 175° C. and about 220° C., between about 240° C. and about 275° C., or between about 310° C. and about 355° C. At least one of A and B can be

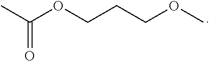

The organic cation can be

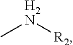

wherein R₂ is H, —CH₂-naphtyl, —CH₂-phenyl, methyl, ethyl, n-propyl, iso-propyl, butyl, n-butyl, sec-butyl, or iso-butyl.

A polymer production process comprising:
providing a crystalline polymer formed from at least two monomers independently selected from the group consisting of Formula I, Formula II, and Formula III,
wherein Formula I is

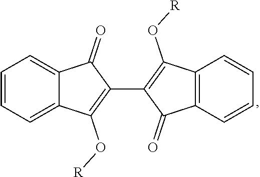

wherein each R is independently a C₁ to C₂₀ linear chain alkyl or a C₁ to C₂₀ branched chain alkyl, wherein any —H maybe substituted with —F,
wherein Formula II is

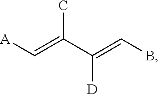

wherein A, B, C and D are independently selected from —C(═O)—R, —C(═O)—OR,

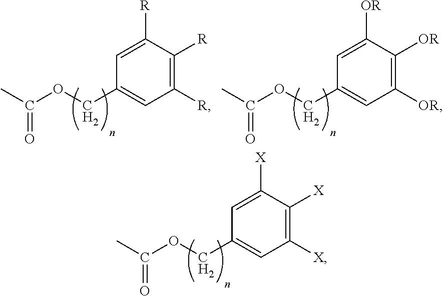

### —C(═S)—R,

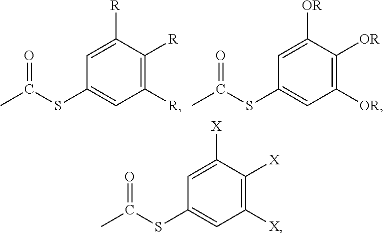

### —C(═O)—NRR,

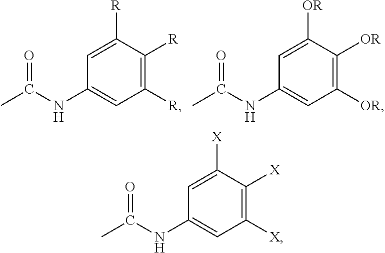

### —C(═O)OM, —S(═O)OM, —S(═O)(═O)OM,

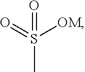

### —NO₂, —OR, —OC(═O)R,

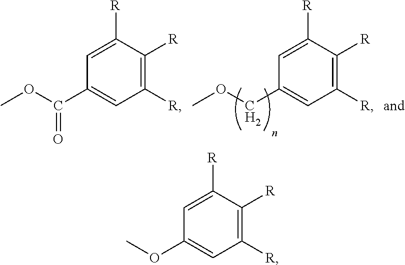

### —H, —F, —Cl, and —CH₃,

wherein each R is independently H, —NO₂, a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein X is a halogen or hydrogen, at least one X is a halogen, M is an organic or inorganic cation, and n=0 to 20 and
wherein Formula III is

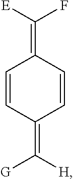

wherein each E, F, G, and H is independently —C(C═O)OR or —CN, wherein each R is independently a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein the monomers are chemically bound to each other by at least one carbon-carbon single bond to form the crystalline polymer;
mixing the crystalline polymer with a solvent to form a mixture;
sonicating the mixture to form a suspension; and
vacuum filtering and heat pressing the suspension. The carbon-carbon single bond can have a bond length between about 1.57 Å and about 1.70 Å. The polymer can have a depolymerization temperature between about 125° C. to about 375° C. The solvent can be an organic solvent. The solvent can be acetone. The solvent can be chloroform. Ultrasound can be performed at an amplitude of about 20, about 4 second pulse and about 1 second rest. Ultrasound can be performed at an amplitude of about 15, about 4 second pulse and about 1 second rest. Ultrasound can be performed at a temperature below 0° C. A and B can be

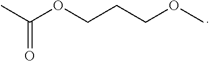

The organic cation can be

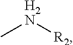

wherein R₂ is H, —CH₂-naphtyl, —CH₂-phenyl, methyl, ethyl, n-propyl, iso-propyl, butyl, n-butyl, sec-butyl, or iso-butyl.

A thermoplastic production process comprising:
providing a crystalline polymer formed from at least two monomers independently selected from the group consisting of Formula I, Formula II, and Formula III,
wherein Formula I is

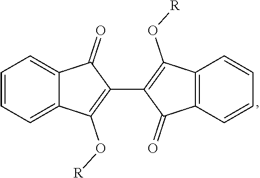

wherein each R is independently C₁ to C₂₀ linear chain alkyl or a C₁ to C₂₀ branched chain alkyl, wherein any —H may be substituted with —F,
wherein Formula II is

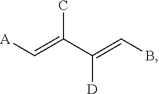

wherein A, B, C and D are independently selected from —C(═O)—R, —C(═O)—OR,

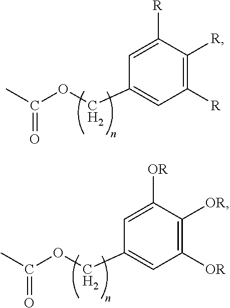

### —C(═S)—R,

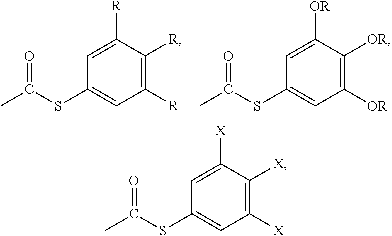

### —C(═O)NRR,

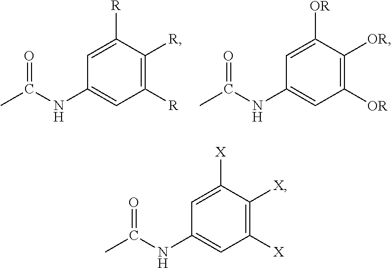

### —C(═O)OM, —S(═O)OM, —S(═O)(═O)OM,

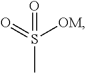

### —NO₂, —OR, —OC(═O)R,

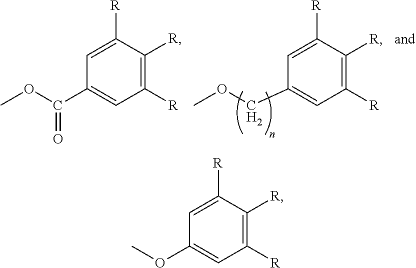

### —H, —F, —Cl, and —CH₃,

wherein each R is independently H, —NO₂, a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein X is a halogen or hydrogen, at least one X is a halogen, M is an organic or inorganic cation, and n=0 to 20, and
wherein Formula III is

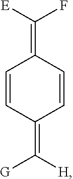

wherein each E, F, G, and H is independently —C(C═O)OR or —CN, wherein each R is independently a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein the monomers are chemically bound to each other by at least one carbon-carbon single bond to form the crystalline polymer;
mixing the crystalline polymer with a first solvent to form a mixture;
subjecting the mixture to heat to form a viscous polymer solution;
mixing the viscous polymer solution with a second solvent to form a residue; and
drying the reside. The carbon-carbon single bond can have a bond length between about 1.57 Å and about 1.70 Å. The polymer can have a depolymerization temperature between about 125° C. to about 375° C. A and B are

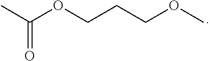

A method of depolymerization comprising:
providing a crystalline polymer formed from at least two monomers independently selected from the group consisting of Formula I, Formula II, and Formula III,
wherein Formula I is

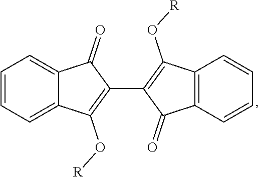

wherein each R is independently a C₁ to C₂₀ linear chain alkyl or a C₁ to C₂₀ branched chain alkyl, wherein any —H maybe substituted with —F,
wherein Formula II is

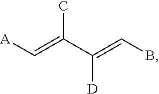

wherein A, B, C and D are independently selected from —C(═O)—R, —C(═O)—OR,

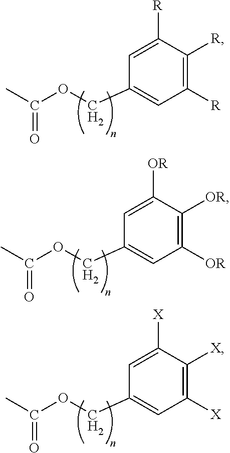

### —C(═S)—R,

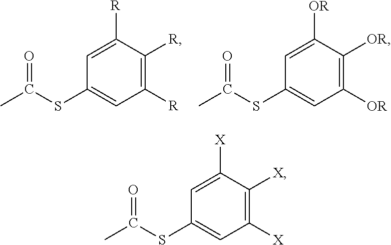

### —C(═O)—NRR,

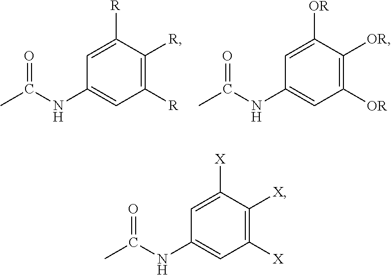

### —C(═O)OM, —S(═O)OM, —S(═O)(═O)OM,

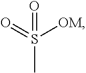

### —NO₂, —OR, —OC(═O)R,

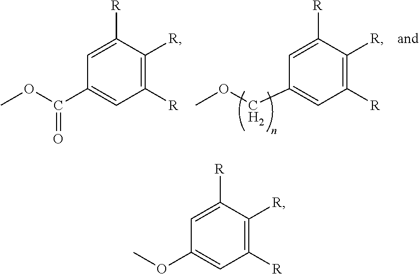

### —H, —F, —Cl, and —CH₃,

wherein each R is independently H, —NO₂, a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein X is a halogen or hydrogen, at least one X is a halogen, M is an organic or inorganic cation, and n=0 to 20, and
wherein Formula III is

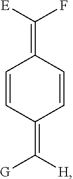

wherein each E, F, G, and H is independently —C(C═O)OR or —CN, wherein each R is independently a C₁ to C₂₀ branched chain alkyl or a C₁ to C₂₀ straight chain alkyl, optionally substituted with —O—, —NRR or —S—, and any hydrogen can be substituted with —F,
wherein the monomers are chemically bound to each other by at least one carbon-carbon single bond to form the crystalline polymer,
wherein the carbon-carbon single bond has a bond length between about 1.57 Å and about 1.70 Å,
mixing the crystalline polymer with a solvent to form a mixture,
heating the mixture to a temperature between about 125° C. to about 375° C., and
recovering the monomers. The temperature can be between about 240° C. and about 275° C. or between about 310° C. and about 355° C. A and B can be

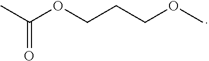

The organic cation can be

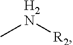

wherein R₂ is H, —CH₂-naphtyl, —CH₂-phenyl, methyl, ethyl, n-propyl, iso-propyl, butyl, n-butyl, sec-butyl, or iso-butyl. The carbon-carbon single bond can have a bond length between about 1.57 Å and about 1.70 Å between about 1.59 Å and about 1.67 Å or between about 1.61 Å and about 1.63 Å.

### BRIEF DESCRIPTION OF THE DRAWINGS

FIG. 1 shows the depolymerization of polyBIT derivatives (A), polyME derivatives (B) and polyQDM derivatives (C).

FIG. 2 shows a universal relationship between the C—C bond lengths (d_{C—C}) and their bond dissociation energies (BDE).

FIG. 3 is the single crystal X-ray diffraction data of polymers and the corresponding depolymerization conditions and yields with values in parentheses for cell lengths, cell angles, and d_{C—C} are the standard deviations.

FIGS. 4A and 4B are the thermogravimetric analysis curves of all 12 polymers with temperatures at 95% TG shown beside each polymer name.

FIGS. 5A and 5B are the Raman spectra of all 12 polymers, including the selected three representative polymers (polyBIT-8, polyME-OMeB and polyQDM-Me) with their corresponding monomers with Raman peak positions significantly different from their polymers.

FIG. 6 is the Differential Scanning Calorimetry (DSC) profiles of all photopolymerized polymer derivatives at high temperature.

FIG. 7A shows photographs of the fresh BIT-6 monomer crystals, polymer crystals, and recycled monomers showing the complete monomer-to-monomer cycle and

FIG. 7B shows overlays of ¹H NMR spectra of BIT-6 fresh monomers (top) and recycled monomers via solution-processed method (bottom) (25° C., CDCl₃).

FIG. 8 is a summary of monomer (monomer isomers for polyME) recovery yields for polymers with different treatments.

FIG. 9 shows (A) representative engineering tensile stress-strain curves of sonicated-processed polyME-OMeB films before and after heat pressing, (B) Raman spectra of polyME-OMeB polymer crystals and sonicated processed films, (C) representative engineering tensile stress-strain curves of polyME-CB films before and after heat pressing (average Young's modulus E=0.81 GPa for heat-pressed film), (D) a representative engineering tensile stress-strain curve of heat-pressed polyBIT-8 film (E=0.12 GPa), (E) a representative engineering tensile stress-strain curve of a commercial nylon filtration membrane (Whatman membrane filters nylon, pore size 0.45 m, E=0.11 GPa), and (F) cyclic tensile-strain tests for determination of the elastic regime of sonicated-processed polyME-OMeB film.

FIG. 10 illustrates a typical load versus displacement plot of polyME-OMeB film and a schematic of the nanoindentation experiments and a summary of Young's Modulus and hardness of polyME-OMeB film estimated by nanoindentation tests and tensile stress-strain tests.

FIG. 11 shows X-ray diffraction profiles of the heat-molded polymer, hot-solution processed polymer, and simulated profile from single crystal data.

FIG. 12 shows the differential scanning calorimetry cyclic plots of (A) polyME-OMeB crystals, (B) the solution-processed polyME-OMeB solid, and (C) the sonication-processed polyME-OMeB polymer film.

FIG. 13 shows representative size exclusion chromatography elution profiles of the solution-processed polyME-OMeB polymer.

FIG. 14 shows representative tensile stress-strain curves of hot-solvent processed polyME-OMeB extruded filaments.

FIG. 15 illustrates the tensile stress-strain curves (A) of sonication-processed polyME-OMeB films after UV, acid, base and microwave, with statistics of Young's modulus measurements (B) for the polyME-OMeB films under different treatments.

FIG. 16 is a table of density functional theory (DFT) calculated Bond Dissociation Energies (BDEs) in kcal/mol.

FIG. 17 is a table of the enthalpies, electronic energies, and corrections (au) for small molecules.

FIG. 18 is a table of the enthalpies, electronic energies, and corrections (au) for common polymers.

FIG. 19 is a table of the enthalpies, electronic energies, and corrections (au) for topochemical polymers.

FIG. 20 is a table of the statistics of the tensile stress-strain tests of polyME-OMeB films.

### DETAILED DESCRIPTION

The present disclosure may be understood more readily by reference to the following description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Certain features of the invention which are, for clarity, described herein in the context of separate embodiments, may also be provided in combination in a single embodiment. Conversely, various features of the invention that are described in the context of a single embodiment, may also be provided separately or in any sub-combination. Further, reference to values stated in ranges include each and every value within that range.

Provided is a crystalline polymer comprising:
at least two monomers, which can be the same or different,
the at least two monomers are chemically bound by at least one carbon-carbon single bond to form the crystalline polymer,
wherein the carbon-carbon single bond has a bond length between about 1.57 Å and about 1.70 Å, and
wherein the polymer has a depolymerization temperature between about 125° C. to about 375° C. The bond length can be between about 1.59 Å and about 1.67 Å or between about 1.61 Å and about 1.63 Å. The depolymerization temperature can be between about 175° C. and about 220° C., between about 240° C. and about 275° C., or between about 310° C. and about 355° C.

Further provided is a crystalline polymer comprising:
at least two monomers, independently selected from the group consisting of Formula I, Formula II, and Formula III,
wherein Formula I is

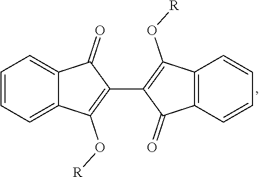

wherein each R is independently a C₁ to C₂₀ linear chain alkyl or a C₁ to C₂₀ branched chain alkyl, in which any —H may be substituted with —F,
wherein Formula II is

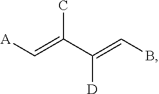

wherein A, B, C and D are independently selected from —C(═R)—R, —C(═O)—OR,

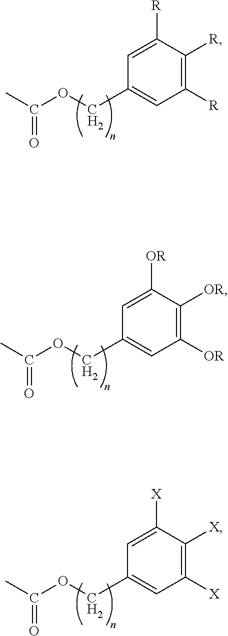

### —C(═S)—R,

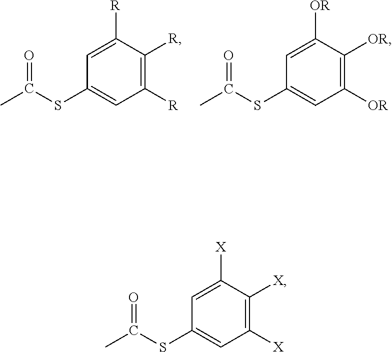

### —C(═O)—NRR,

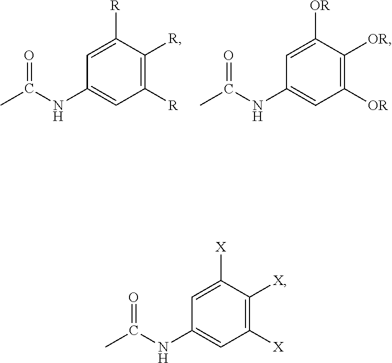

### —C(═O)OM, —S(═O)OM, —S(═O)(═O)OM,

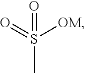

### —NO₂, —OR, —OC(═O)R,

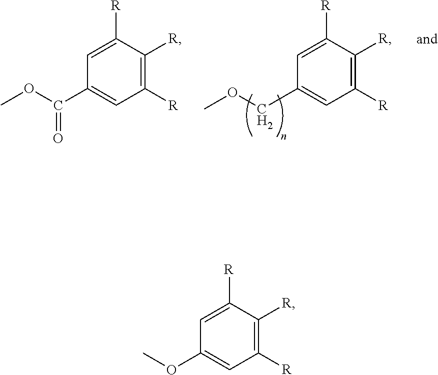

### —H, —F, —Cl, and —CH₃,

wherein each R is independently H, —NO₂, a C₁ to C₂₀ branched chain alkyl, or a C₁ to C₂₀ straight chain alkyl, wherein the branched chain alkyl or the straight chain alkyl is optionally substituted with —O—, —NRR or —S—, and any hydrogen can be substituted with —F,
wherein X is a halogen or hydrogen, at least one X is a halogen, M is an organic or inorganic cation, and n=0 to 20 and
wherein Formula III is

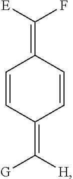

wherein each E, F, G, and H is independently —C(C═O)OR or CN, wherein each R is independently a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein the monomers are chemically bound to each other by at least one carbon-carbon single bond to form the crystalline polymer. The carbon-carbon bond can have a bond length between about 1.57 Å and about 1.70 Å, between about 1.59 Å and about 1.67 Å or between about 1.61 Å and about 1.63 Å. The polymer can have a depolymerization temperature between about 125° C. to about 375° C., between about 175° C. and about 220° C., between about 240° C. and about 275° C., or between about 310° C. and about 355° C. At least one of A and B can be

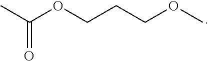

The organic cation can be

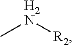

wherein R₂ is H, —CH₂-naphtyl, —CH₂-phenyl, methyl, ethyl, n-propyl, iso-propyl, butyl, n-butyl, sec-butyl, or iso-butyl.

The crystalline polymers are derived from a combination of monomers of Formula I, II or III.

The crystalline polymer can include monomers of Formula I. Formula I is defined as:

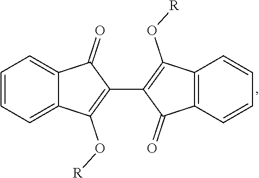

wherein each R is independently a C₁ to C₂₀ linear chain alkyl or branched chain alkyl. Any —H on the alkyl group can be substituted with fluorine. The entire alkyl chain can be fluorinated.

The crystalline polymer can include monomers of Formula II. Formula II is defined as:

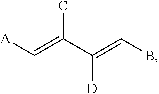

wherein A, B, C and D are independently selected from —C(═O)—R, —C(═O)—OR,

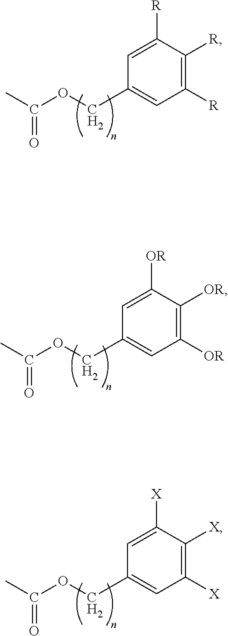

### C(═S)—R,

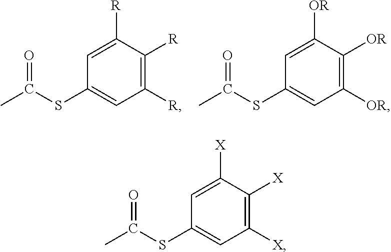

### —C(═O)—NRR,

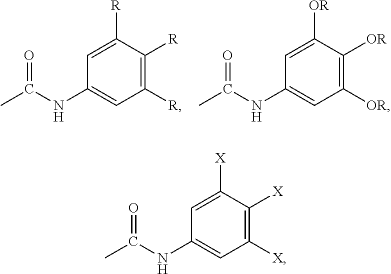

### —C(═O)OM, —S(═O)OM, —S(═O)(═O)OM,

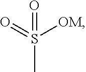

### —NO₂, —OR, —OC(═O)R,

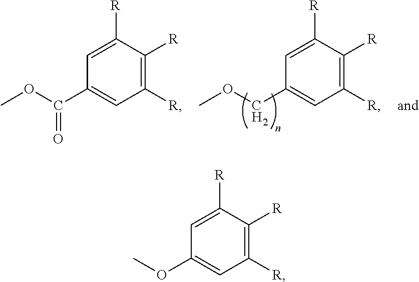

### —H, —F, —Cl, or —CH₃,

wherein each R is independently H, —NO₂, a C₁ to C₂₀ branched chain alkyl or straight chain alkyl, optionally substituted with —O—, —NRR or —S—, and any hydrogen can be substituted with —F, wherein X is a halogen or hydrogen, at least one X is a halogen, M is an organic or inorganic cation, and n=0 to 20.

Examples of organic cations include ammonium, monoalkyl ammonium, dialkyl ammonium, substituted and unsubstituted imidazolium, substituted and unsubstituted pyridinium, and substituted and unsubstituted phosphonium. The substituted organic cations can contain functional groups including ketones, esters, thiocarbonyls, thioesters, ethers, thiols, additional amino groups, nitro, and halogens.

The organic cation can be an ammonium derivative of the Formula

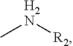

wherein R₂ is H, C₁ to C₂₀ alkyl, or C₁ to C₂₀ substituted alkyl. Examples of alkyl include, but are not limited to —CH₂-naphtyl, —CH₂-phenyl, methyl, ethyl, n-propyl, iso-propyl, butyl, n-butyl, sec-butyl, or iso-butyl. The alkyl group can contain one or more substituents, which can be the same or different, such as a substituent selected from a ketone, an ester, a thiocarbonyl, a thioester, an ether, a thiol, an additional amino group, a nitro, and a halogen.

Examples of inorganic cations include, but are not limited to, lithium, sodium, potassium, beryllium, magnesium, calcium, strontium, barium, scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, and mercury.

At least one of A and B can be an ester wherein the alkyl group contains at least one ether linkage, thiol linkage, or amine linkage. In an embodiment A and B are

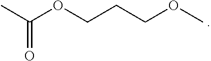

In another embodiment A and B are

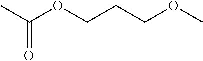

with C and D as hydrogen.

The crystalline polymer can include monomers of Formula III. Formula III is defined as:

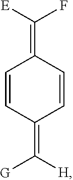

wherein each E, F, G, and H is independently —C(C═O)OR or —CN, wherein each R is independently a C₁ to C₂₀ branched chain alkyl or a C₁ to C₂₀ straight chain alkyl, wherein the branched chain alkyl or the straight chain alkyl can be optionally substituted with one or more substituents, which can be the same or different, and which can be selected from —O—, —NRR and —S—, and any hydrogen can be substituted with —F.

The crystalline polymer contains at least two monomers. The monomers can be the same or different. The monomers are chemically bound together by a carbon-carbon single bond.

The resulting bond lengths between the monomers in the crystalline polymer range from about 1.57 Å and about 1.70 Å (such as 1.57 Å to about 1.70 Å, about 1.57 Å to 1.70 Å, or 1.57 Å to 1.70 Å), or between about 1.59 Å and about 1.67 Å (such as 1.59 Å to about 1.67 Å, about 1.59 Å to 1.67 Å, or 1.59 Å to 1.67 Å), or between about 1.61 Å and about 1.63 Å (such as 1.61 Å to about 1.63 Å, about 1.61 Å to 1.63 Å, or 1.61 Å to 1.63 Å).

The depolymerization temperature ranges between 125° C. and 375° C. The depolymerization temperature can be between about 150° C. and about 350° C. (such as 150° C. to about 350° C., about 150° C. to 350° C., or 150° C. to 350° C.), between about 175° C. and about 325° C. (such as 175° C. to about 325° C., about 175° C. to 325° C., or 175° C. to 325° C.), between about 200° C. and about 300° C. (such as 200° C. to about 300° C., about 200° C. to 300° C., or 200° C. to 300° C.), and between about 225° C. and about 275° C. (such as 225° C. to about 275° C., about 225° C. to 275° C., or 225° C. to 275° C.). Depolymerization temperatures include between about 175° C. and about 220° C. (such as 175° C. to about 220° C., about 175° C. to 220° C., or 175° C. to 220° C.), between about 240° C. and about 275° C. (such as 240° C. to about 275° C., about 240° C. to 275° C., or 240° C. to 275° C.), or between about 310° C. and about 355° C. (such as 310° C. to about 355° C., about 310° C. to 355° C., or 310° C. to 355° C.).

Any combination of monomers of Formulae I, II and III can be used to produce the desired crystalline polymer with elongated carbon-carbon single bonds. For example, a crystalline polymer can be formed by combining the same or different monomers of Formula I, combining the same or different monomers of Formula II, or combining the same or different monomers of Formula III. Additionally, the crystalline polymer can be formed from a combination of monomers of Formula I and Formula II, Formula I and III, Formula II and Formula III, or Formulae I, II, and III.

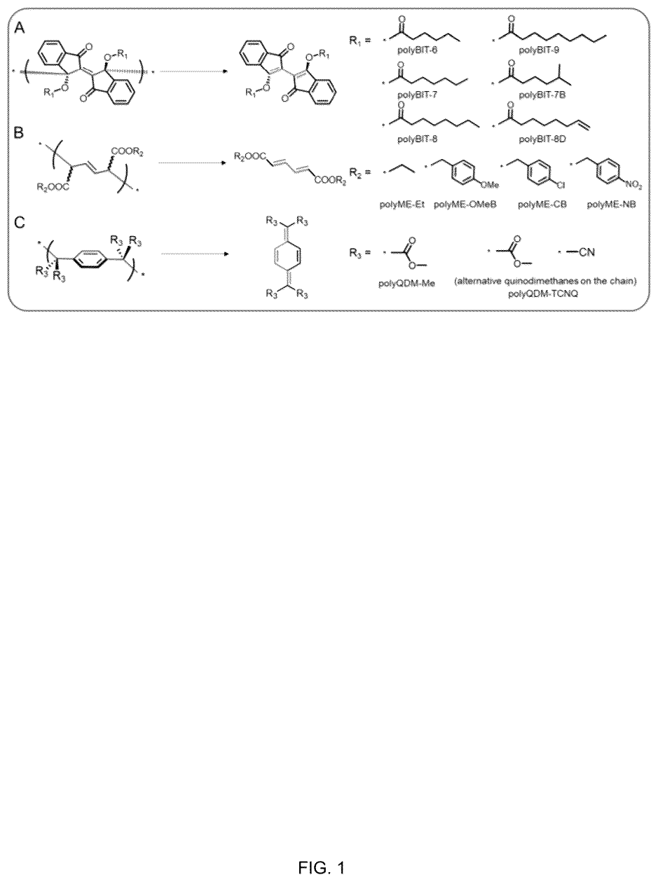

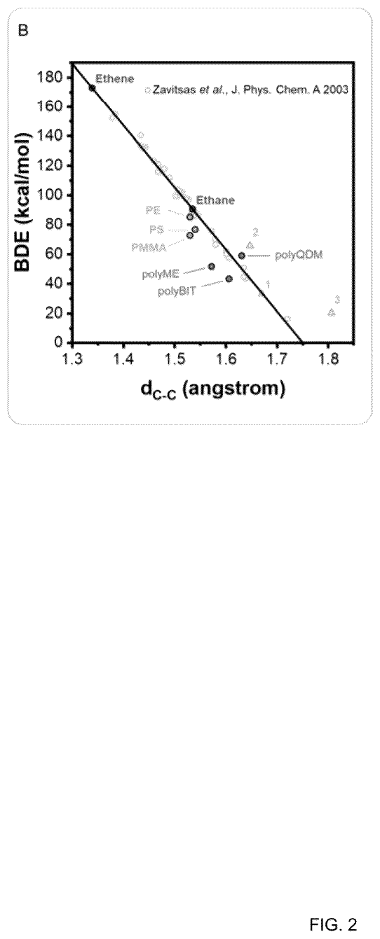

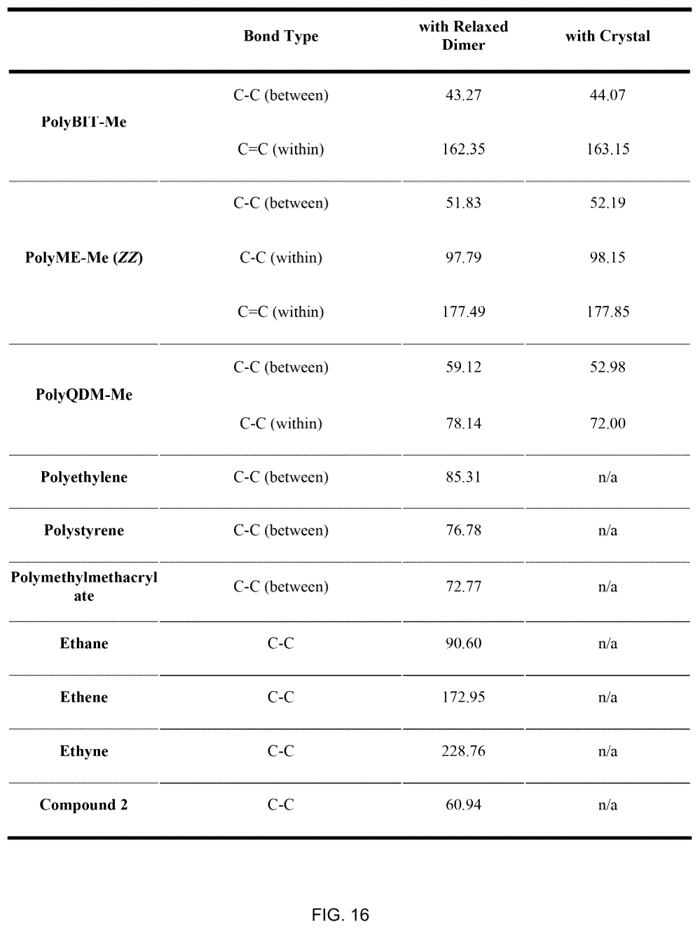

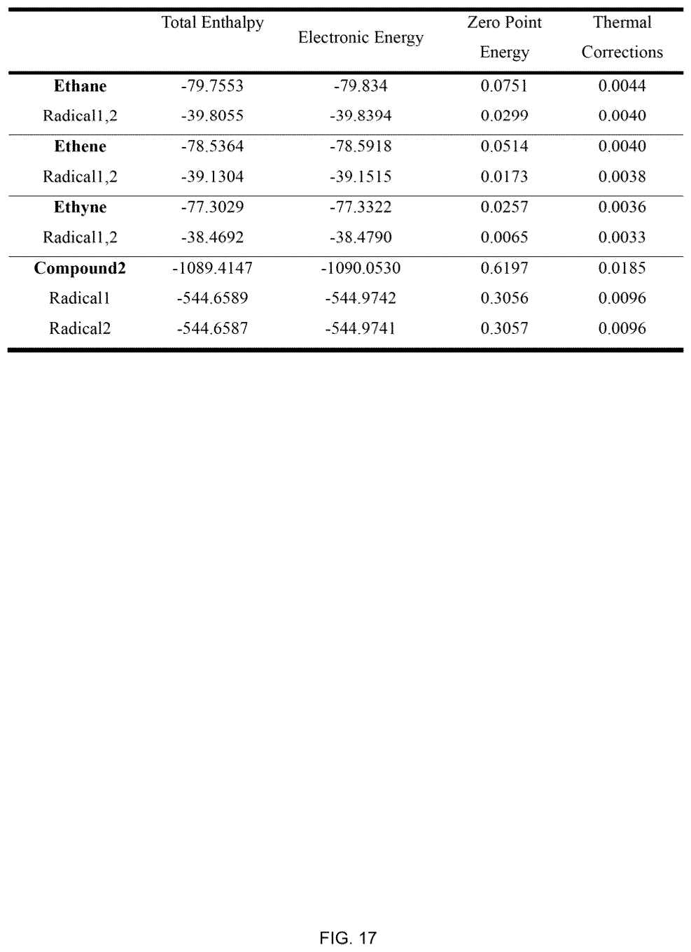

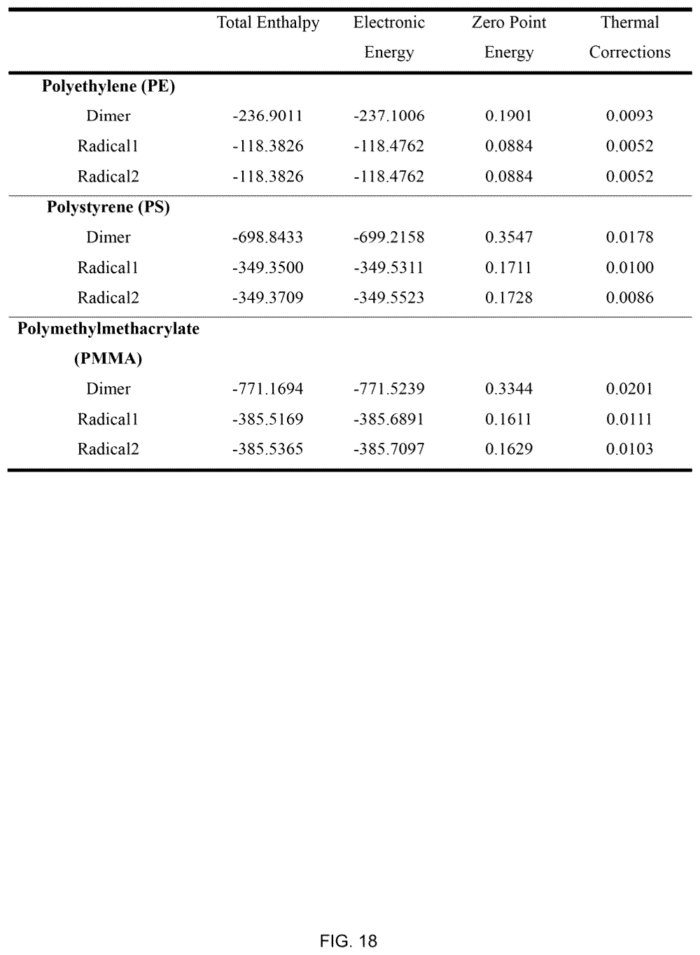

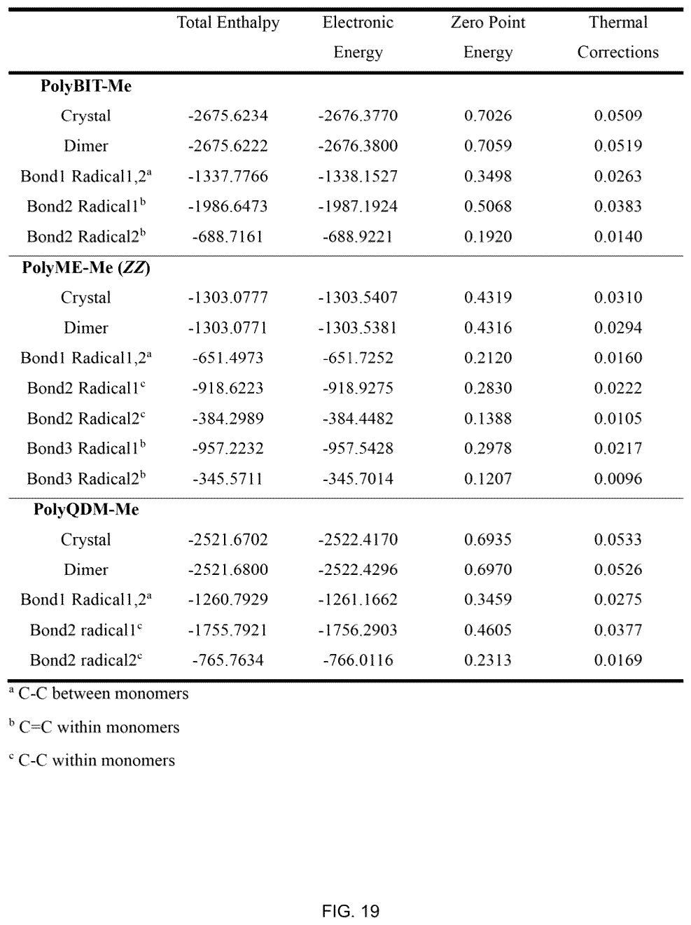

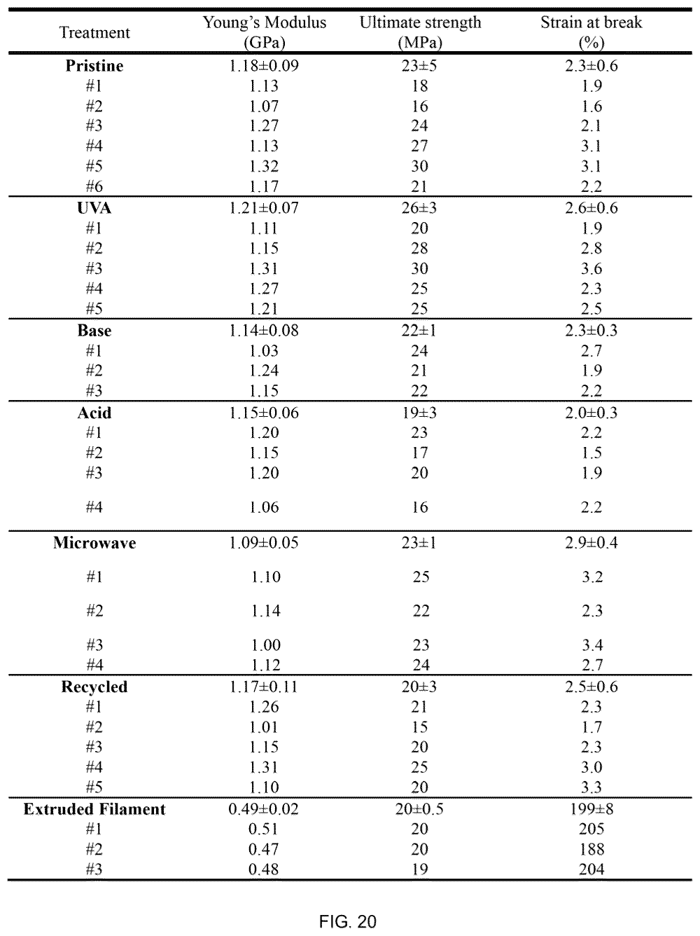

Three photo-polymerized hydrocarbon backbones featuring elongated C—C bonds, namely polybiidenedionediyl (polyBIT), polymuconate (polyME) and polyquinodimethane (polyQDM) derivatives (FIG. 1), exemplify the disclosure. To reveal the relationship between C—C bond length and bond dissociation energy (BDE) in these polymer systems, as well as to elucidate the role of the distinct monomer cores on the depolymerization activity, the bond strengths of all cleavable backbone bonds within the associated polymers were characterized using density functional theory (DFT). These characterizations were performed on oligomers of each polymer using geometries obtained from representative polymer crystal structures, and the bond energies were calculated as the enthalpy difference between these structures and the radicals associated with the indicated bond scissions. All DFT parameters and results are summarized in FIGS. 16, 17, 18 and 19. The simulation was validated by calculating the BDE of a few known molecules such as ethane, ethene, and compound 2. Then conventional polymers PE, PS and PMMA were characterized in comparison with the three topochemically polymerized backbones. It was observed that the trend of BDE and C—C bond distances of all six polymer backbones agrees well with the quasi-linear relationship (FIG. 2). The three topochemical backbones exhibit relatively weak bonds (i.e., ranging from ˜40 to ˜50 kcal/mol) at the sterically crowded position between two monomers (FIG. 2), while PE, PS and PMMA exhibit much stronger bonds (˜80 to ˜90 kcal/mol) (FIG. 2). This provides the hypothesized role of the elongated bonds resulting from topochemical depolymerization. We also observe that the other bonds in the polymer backbone, which could in principle also be thermally cleaved, are much higher in energy and comparable to typical C—C (˜90 kcal/mol) and C═C (˜170 kcal/mol) bonds in unsubstituted alkanes and alkenes; respectively. This provides a prediction that thermal cleavage of these systems can be highly selective, because in each case, the elongated bond is well-separated in bond energy from the other carbon-carbon bonds. Interestingly, the predicted BDE of the elongated bond in polyQDM derivatives is slightly higher than that of polyBIT and polyME derivatives, despite their longer C—C bonds. This deviation reveals the role played by concomitant structural rearrangement and local chemical environment of C—C bonds, likely the destruction of aromaticity in this case, in the calculated bond energies.

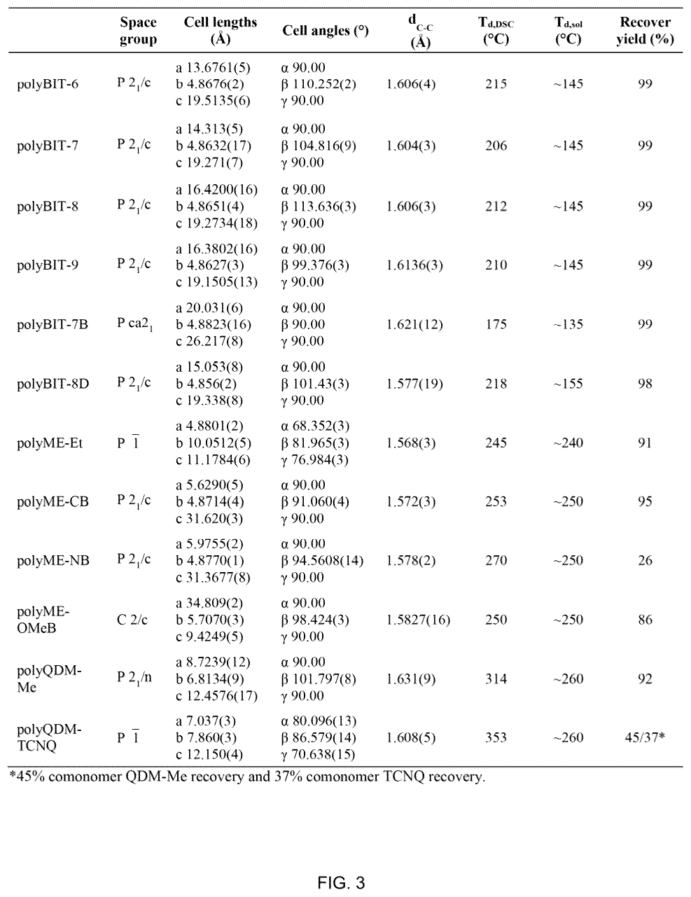

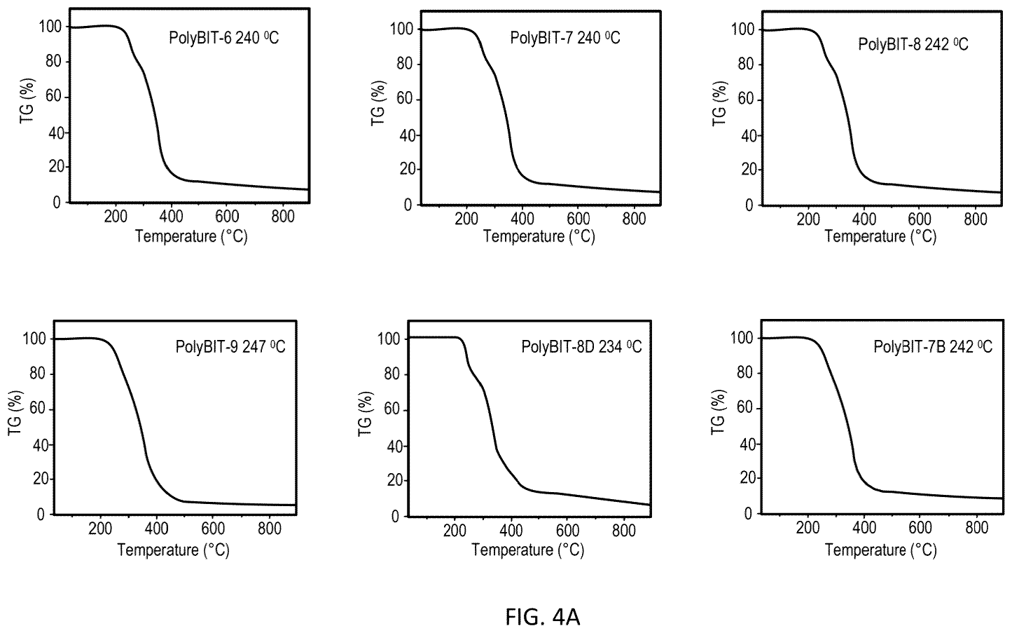

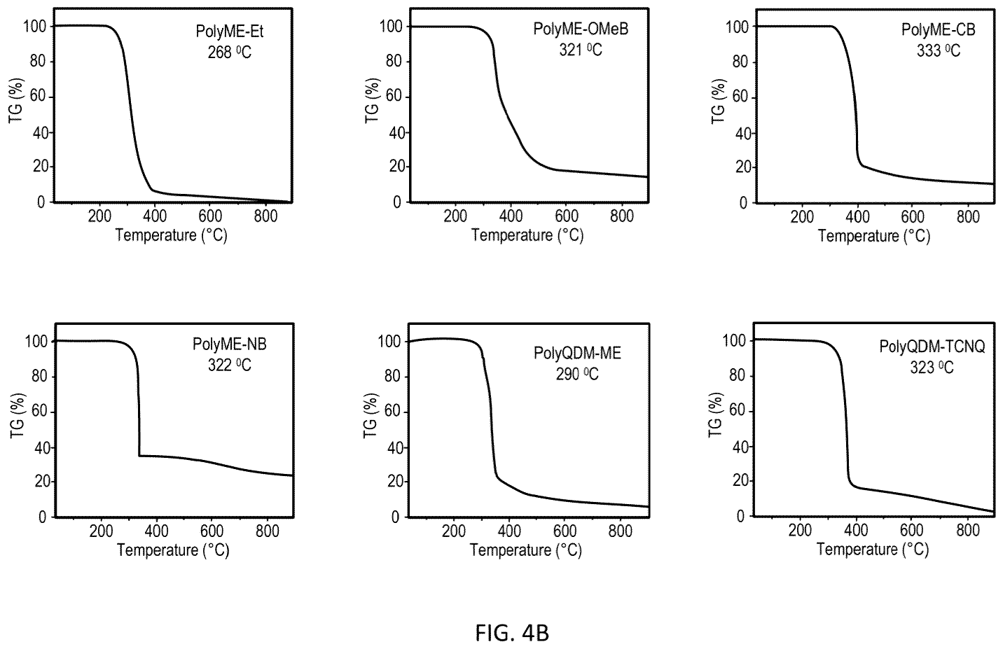

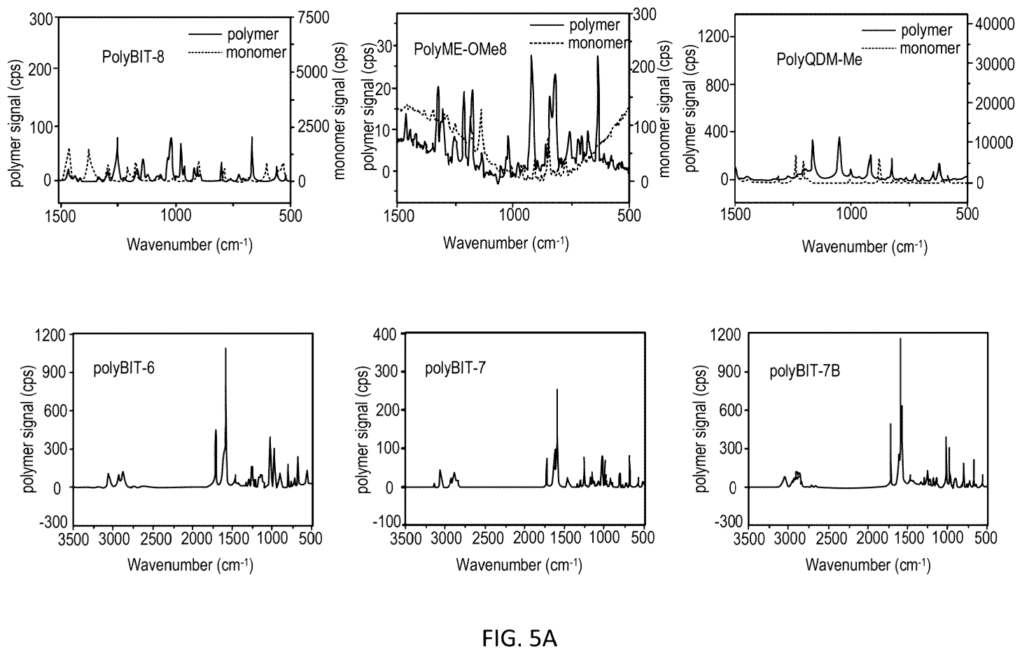

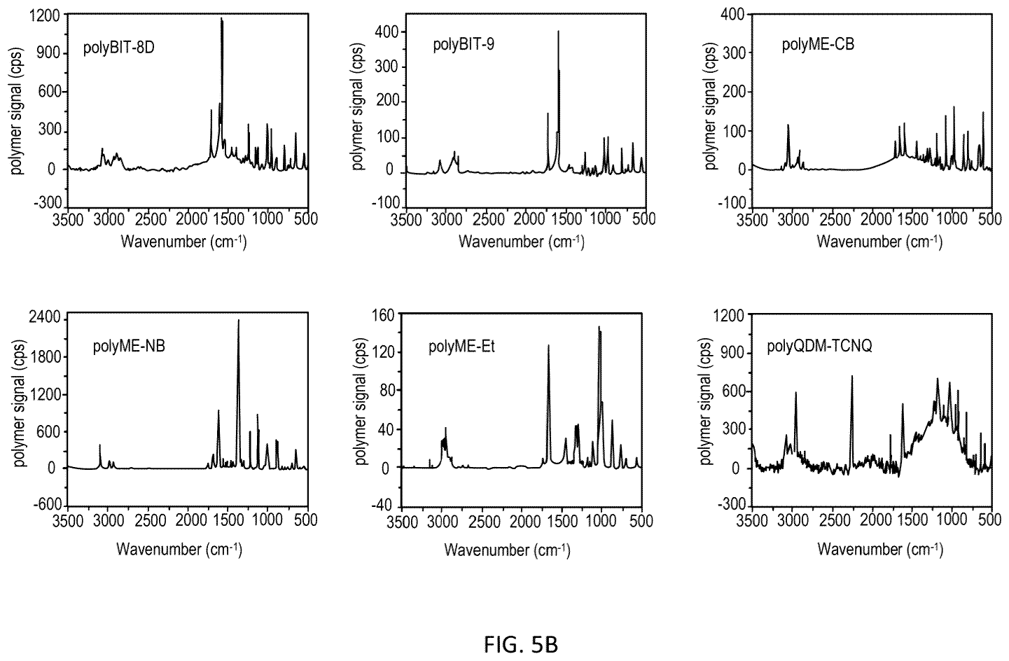

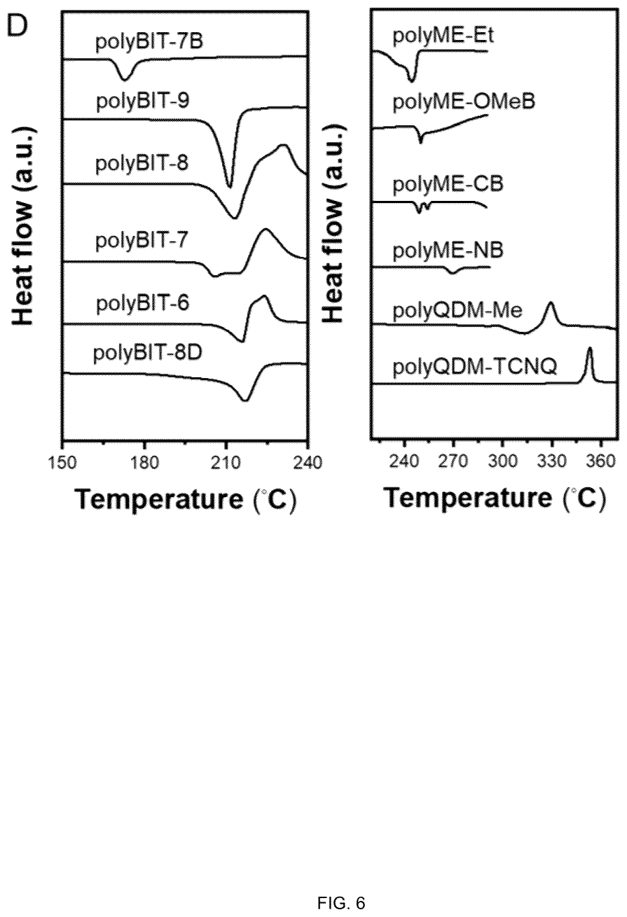

Single crystal structures and C—C bond lengths are summarized in FIG. 3. Thermal stability of the polymers was characterized using thermogravimetric analysis (TGA) to ensure depolymerization reaction is carried out below the decomposition temperature (FIGS. 4A and 4B). Raman spectroscopy was deployed to characterize the elongated C—C bonds (FIGS. 5A and 5B). Unfortunately, the stretching modes of the elongated C—C bonds in these polymers were complicated by the C—O stretching, C—O bend and aryl ring deformation at 800-1200 cm¹ range and, therefore, the exact individual vibration modes could not be assigned. Even though, Raman spectra do provide evidence that polymer absorption is significantly different from its corresponding monomer absorption. Differential Scanning Calorimetry (DSC) was used to characterize the possible solid-state depolymerization reactions for all 12 polymer crystals, and the results are summarized in FIG. 3 and FIG. 6. Generally, solid-state depolymerization of polyBIT, polyME, and polyQDM polymers occur at 175˜218° C., 245˜270° C., and 314˜353° C.; respectively.

The polymers generally follow a negative correlation between the depolymerization temperature and C—C bond lengths within each group, indicating the C—C bond length a dominant factor. This is particularly clear when comparing polyBIT-8D, polyBIT-8 and polyBIT-7B, where polyBIT-8D with shortest d_{C—C} of 1.577 Å exhibits highest T_{d} of 218° C., and polyBIT-7B with longest d_{C—C} of 1.621 Å exhibits significantly lower T_{d} of 175° C. Such relationship clearly shows that longer C—C bonds generated by topochemistry lead to lower depolymerization temperature of the polymers. Similar trends are also observed in the polyQDM system. The systematically higher T_{d} observed in the polyQDM group is fully supported by the DFT characterizations, where polyME and polyBIT derivatives follow the BDE—d_{C—C} trend similar to PE, PS, PMMA, whereas polyQDM has a much higher BDE (FIG. 2). Secondly, it should be noted that side chain chemistries have a secondary effect on the depolymerization temperature. For example, polyBIT-6, polyBIT-7 and polyBIT-8 all have a very similar d_{C—C} of 1.604˜ 1.606 Å but they exhibit slightly different T_{d} (varying from 206 to 215° C.). Also, in polyME system where the bond length variations are quite small (within ˜0.01 Å), less bulky and less polar ethyl group (-Et) leads to a slightly lower T_{d} and strongly polar and rigid nitro group (—NB) leads to a slightly higher T_{d} as compared with other polyME derivatives. Nevertheless, the deviation in each polymer system is relatively small. These observations suggest that the side chain effect provides us an extra knob to fine-tune the polymer depolymerization properties tailorable for different practical applications.

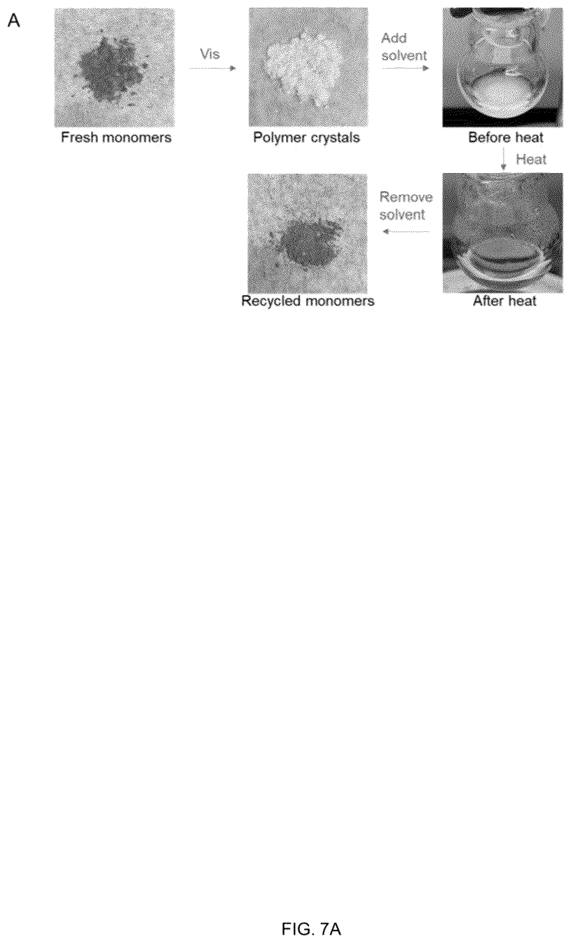

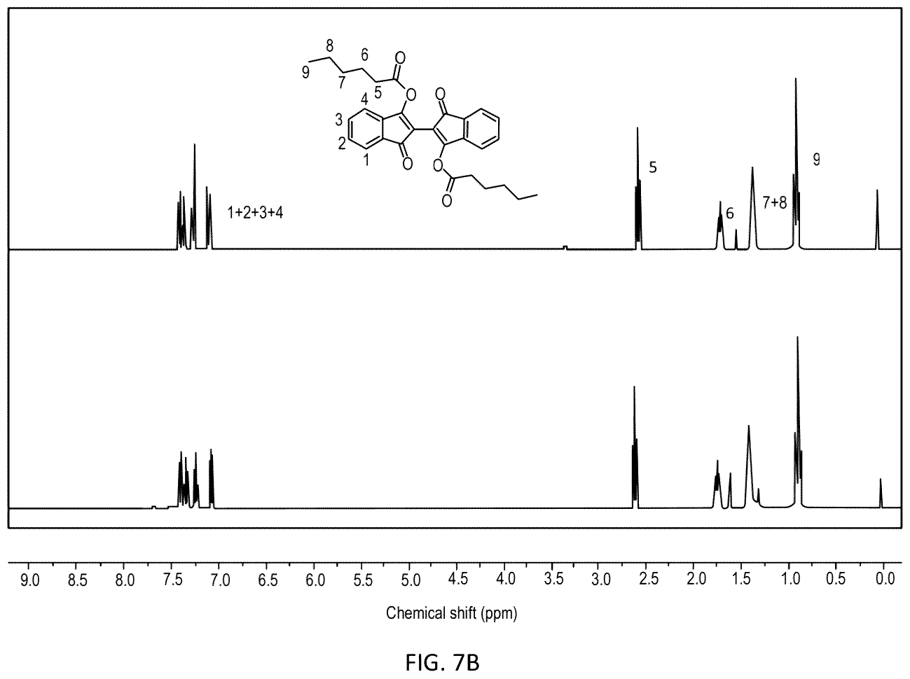

The solid-state depolymerization provides important fundamental insights into the reaction dynamics, but the actual recovery yield for the solid-state reaction is typically low because it is challenging to heat the large amount of polymer solids uniformly and local overheating can lead to polymer/monomer decomposition and by-product formation. To address this issue, it was found that adding a small amount of solvent to dilute the released monomer is practically helpful to increase the recovery yield and lower down the depolymerization temperature (FIG. 3). For example, polyBIT-6 (with hexanoyl as the Ri group) undergoes near quantitative depolymerization when suspended in a high-boiling point organic solvent, anisole, under a mild temperature of 145° C. within 20 minutes. The light-yellow polyBIT-6 polymer crystals were converted to orange monomer powder with over 99% yield. This solution depolymerization method was later expanded to all six polyBIT polymers, providing orange monomers with near quantitative yields ranging from 98% to 99% (FIG. 7A and FIG. 7B). Most polyME derivatives start to depolymerize at 230° C. and undergo rapid depolymerization at 240-250° C. using diphenyl ether (DPE) as the solvent. The monomers were obtained as a white powder with yields ranging from 86%-95%. The slightly lower recovery yield of the polyME derivatives is likely limited by the bimolecular Diels-Alder reaction between the as-produced monomers. For polyME-NB, only 26% monomer recovery yield was obtained with insoluble black powder residue remained. The low yield may be attributed to poor solubility and stability of monomer ME-NB.

As implied by BDE and T_{d}, the third exemplary series based on polyQDM indeed require the highest temperature to depolymerize in solvent. The depolymerization of polyQDM-Me in DPE did not happen until 260° C. PolyQDM-Me polymers were converted to light-yellow monomer powders with an excellent yield of 92%. The ˜8% monomer loss was likely due to the reaction between the quinodimethane monomer and oxygen under ambient conditions. The other polymer polyQDM-TCNQ featuring a shorter d_{C—C} is even harder to depolymerize. Only 45% comonomer QDM-Me recovery and 37% comonomer TCNQ were recovered even after 2 hours of heating at over 260° C. in DPE. An even higher temperature may be needed in this case due to the shorter C—C bond, but we are currently limited by the availability of a suitable solvent.

Kinetic experiments for eight representative polymers were conducted to shed light on the depolymerization mechanism (including polyBIT-8, polyBIT-8D, polyBIT-7B, polyME-Et, polyME-CB, polyME-OMeB, polyQDM-Me and polyQDM-TCNQ). Polymers with similar crystal size were used to eliminate the surface area dependence on the reaction kinetics. HPLC was used to determine monomer conversion at different reaction time. For the polyBIT series, the reaction temperature was kept at 150° C. for all cases. The fastest monomer conversion was obtained for polyBIT-7B with the longest C—C bond, while slowest monomer conversion was obtained for polyBIT-8D with the shortest C—C bond. The result correlates well with lowest depolymerization temperature required for polyBIT-7B. For polyME series, the temperature was kept at 240° C. The three polymers did not show obvious difference in terms of conversion rate as the C—C bond lengths are quite similar among these polymers. For polyQDM series at 260° C., the kinetic curves show that polyQDM-TCNQ with shorter C—C bond length (higher BDE) takes much longer to depolymerize, agreeing with our theory. An important fact is that in all these reactions, monomer concentrations increased linearly with time in early stage, and oligomers were not observed throughout the reaction. These suggest an “unzipping” depolymerization mechanism for all three polymer systems. The solution depolymerization experimental results fully support our hypothesis that recyclable hydrocarbon polymers can be obtained via introducing elongated C—C bonds between the adjacent monomer units. It is interesting to note that all three systems discussed here have a 1,3-diene or 1,3,5-triene core imbedded in the monomers, and all of them exhibit relatively high topochemical polymerization reactivity. This suggests that the diene or triene core can be used as a general building block for future polymer design in a vast chemical space.

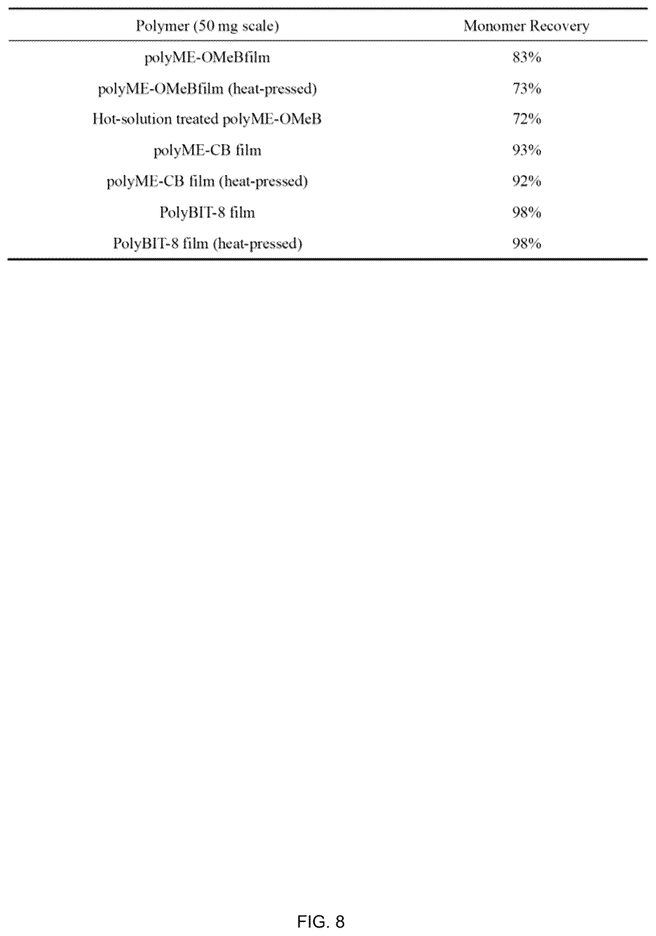

The single crystalline polymers normally do not melt and are difficult to dissolve in common solvents. Processing of these polymers into a useful form is challenging. Here, it was first to introduce a general, simple, and effective sonication method to process these polymers into entangled networks of crystalline fibers and mechanically robust free-standing films. In this work we mainly focused on polyME derivatives for demonstration of their potential practical applications. FIGS. 4A and 4B show the film fabrication process for a polyME derivative, polyME-OMeB. After the ultrasonication process in a solvent (e.g., acetone), the white polymer crystals are dispersed uniformly forming a gel-like suspension. This suspension is stable for many days without exhibiting aggregation or precipitation. Then, a smooth and mechanically flexible thin films with a thickness of ˜0.1 mm was prepared via a simple vacuum-filtration heat-pressing process. This method is generalizable to other polymer crystals as well. For example, PolyME-CB and polyBIT-8 can be processed in the same way, and similar high-quality recyclable films can be obtained. The processed films can be reverted to monomer with similar yields as the pristine crystals (FIG. 8).

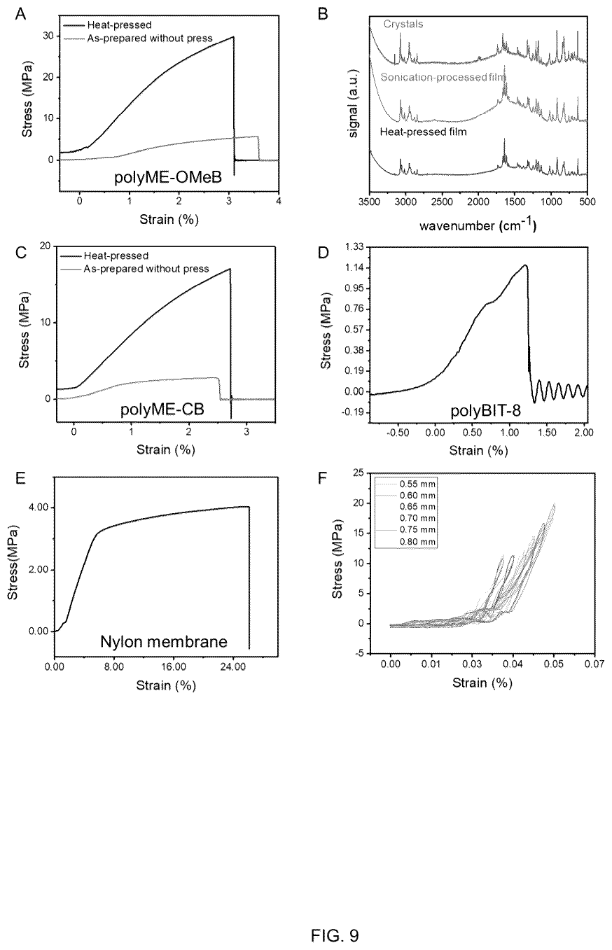

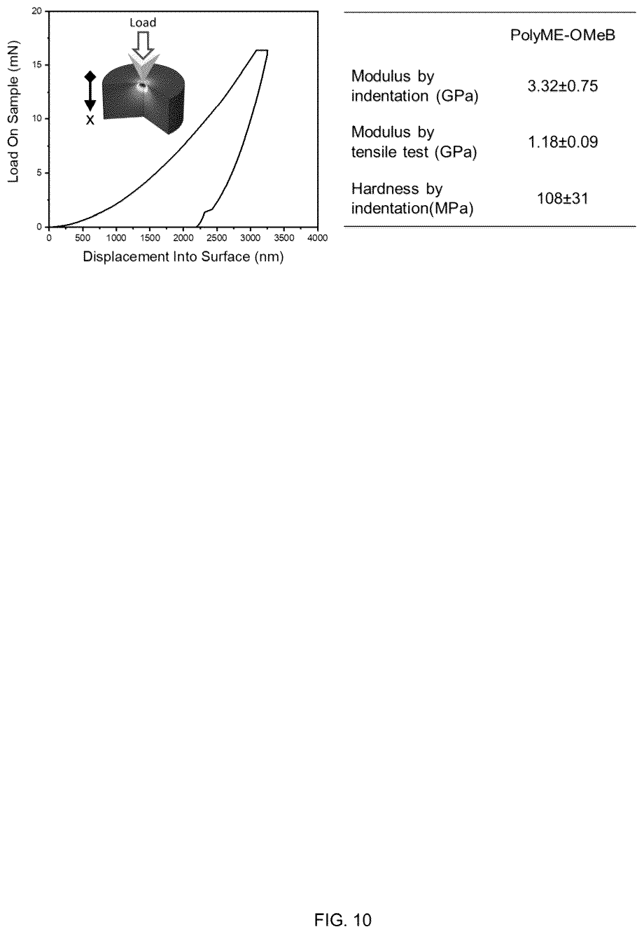

The morphology of the sonication-processed films was characterized using X-ray diffraction (XRD) and scanning electron microscopy (SEM) techniques. The XRD profile of the sonication-processed film exhibited strong crystallinity, suggesting the fibers are crystalline, and the ultrasonication process is not able to dissemble the polymer chains completely. A low magnification SEM image of polyME-OMeB showed that the film is very uniform. A higher magnification SEM image indicated that the film is porous and comprises an interpenetrating network of long fibers with widths of around 20 to 100 nm. This feature is similar to the morphology of a commercial nylon membrane. The mechanical properties of the thin films were characterized using standard tensile tests. The pristine polyME-OMeB film exhibited a large Young's modulus of 1.32 GPa (avg. 1.18 Gpa) and a tensile strength of 30 Mpa (avg. 23 Mpa) (FIG. 9). This is much stronger than a commercial nylon membrane owing to the great crystallinity of the film. Films were prepared from the recycled monomers, and these films exhibited similar tensile stress-strain characteristics. Furthermore, the outstanding mechanical properties of the polyME-OMeB films were further confirmed by indentation measurements (FIG. 10).

The porous nature, the strong mechanical properties and the excellent solvent resistance of the sonication-processed polymer film suggest potential applications in membrane separations. A simple demonstration was conducted using the polyME-OMeB film as a filtration membrane. Specifically, a suspension of a conjugated polymer in methanol was filtrated through a membrane made of polyME-OMeB. Colorless filtrate was collected in the flask while all the solids remained inside the funnel. The dried conjugated polymer flake can be easily peeled off from the recyclable filtration membrane.

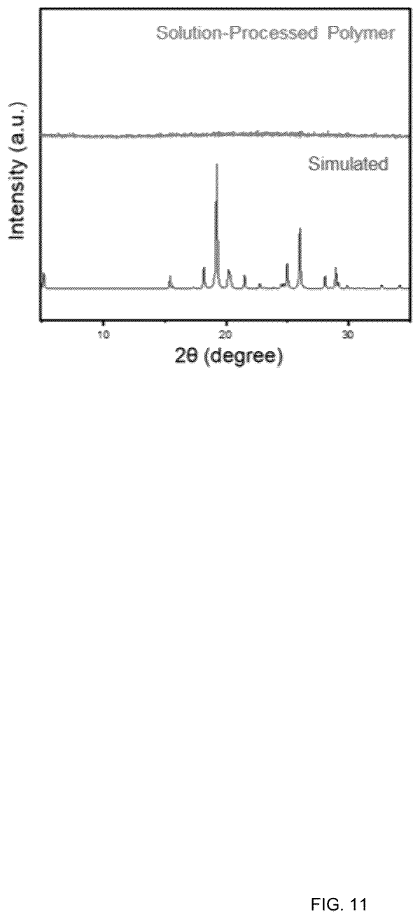

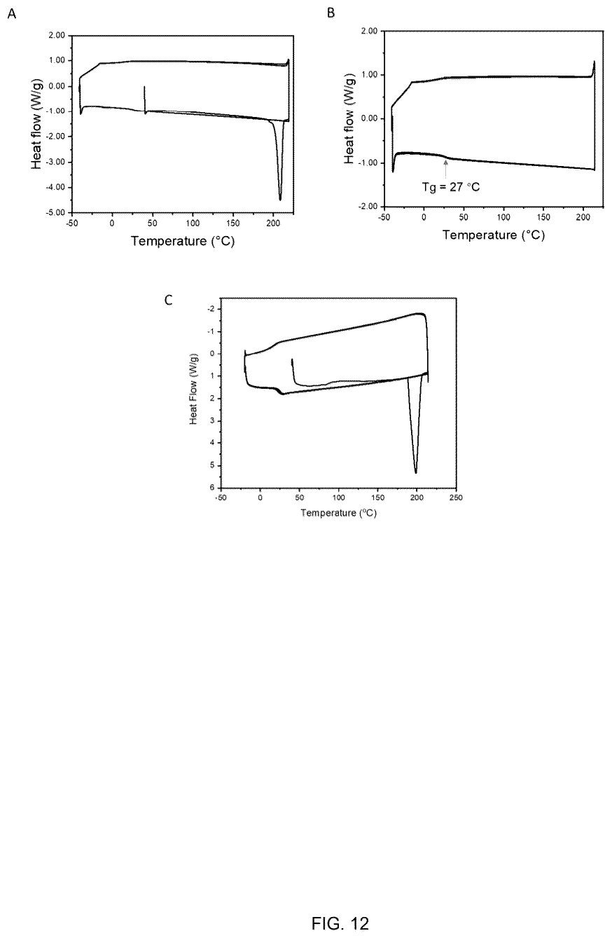

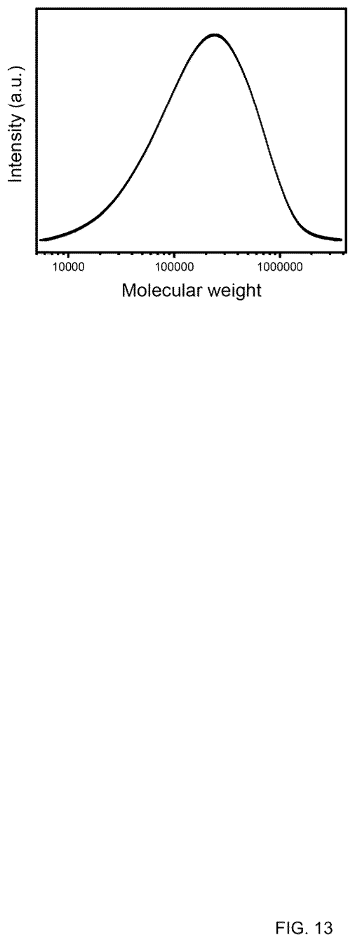

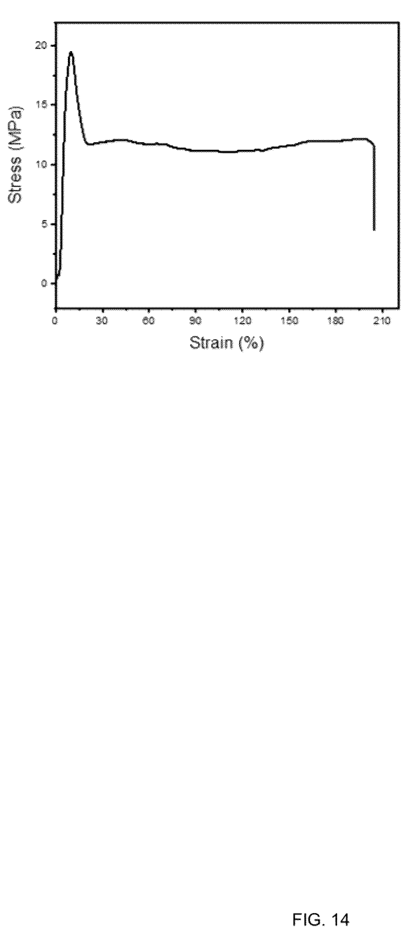

It was further demonstrated that the polymer single crystals can be converted into thermoplastics. For example, it was found that polyME-OMeB crystals, although not soluble at room temperature, indeed dissolves in DPE completely at −180° C. forming a viscous solution. Precipitation from methanol and drying in the vacuum oven give a hard, off-white polymer solid. The XRD profile (FIG. 11) and DSC analysis (FIG. 12) of this hot-solution-processed polymer suggested that it is amorphous with a reversible glass transition temperature at around 27° C. The hot-solution-processed polymers had a number-average molecular weight of 1.1×10⁵ g mol⁻¹ and a polydispersity index of 2.9 (FIG. 13), which is similar to or higher than most reported recyclable polymers. The thermoplastic properties and amorphous nature make the polymer easily processable using conventional techniques such as molding, extrusion, and 3D-printing. Here, we show that polymer filament can be extruded from a microcompounder at 170° C. with a diameter of ˜1.6 mm. The filament was then fed into a 3D printer and a Purdue logo was printed. The modulus and tensile strength of the polymer filament were determined to be 0.51 GPa and 20 MPa (FIG. 14), which are comparable to widely used high-density polyethylene, polypropylene and nylon. This amorphous polymer can be extended to 300% of its original length (200% strain) without breaking. Notably, these high values were achieved without the use of any reinforcement additives such as carbon fibers.

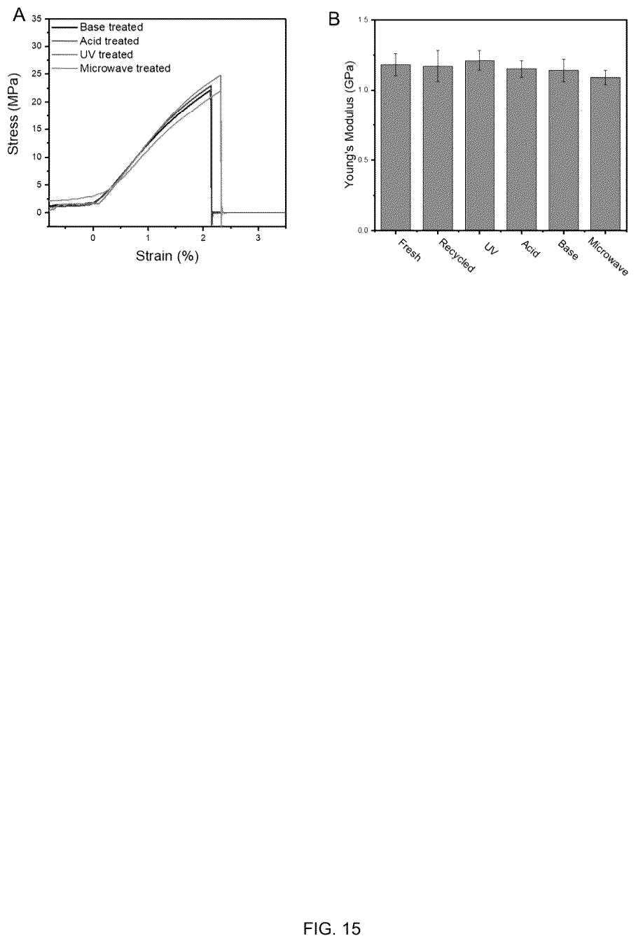

A significant advantage of the studied topochemical polymers in comparison with other recently reported depolymerization motifs is that they are chemically inert and thermally resilient. Sonication-processed PolyME-OMeB polymer films with strong 350 nm UV light (˜900 lux) were treated in air, acid (pH-0) and base (pH-14) for 24 hours and microwaved for 5 min (over 95° C.). No obvious chemical degradation could be detected by examining the polymer and the solvents after these harsh treatments. The tensile stress-strain characteristics of the films after treatments were measured and no obvious degradation in mechanical properties was found (FIG. 15). The statistics of the 30 samples that were measured are shown in FIG. 15, suggesting excellent reproducibility and uniformity of the polymer films. The outstanding chemical and thermal stability makes them potentially useful in a wide range of immediate applications, overcoming the limitations of other types of recyclable polymers, which are unstable in corrosive environments and can only be used at near room temperature.

In view of the above, also provided is a polymer production process comprising:
providing a crystalline polymer formed from at least two monomers independently selected from the group consisting of Formula I, Formula II, and Formula III,
wherein Formula I is

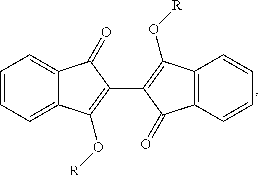

wherein each R is independently a C₁ to C₂₀ linear chain alkyl or a C₁ to C₂₀ branched chain alkyl, wherein any —H maybe substituted with —F,
wherein Formula II is

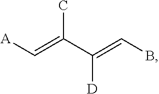

wherein A, B, C and D are independently selected from —C(═O)—R, —C(═O)—OR,

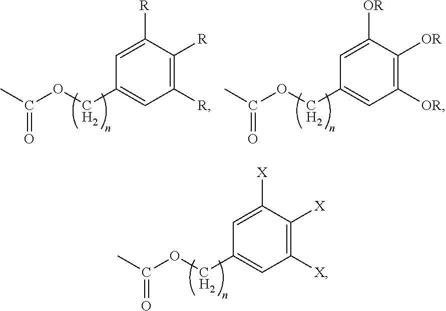

### —C(═S)—R,

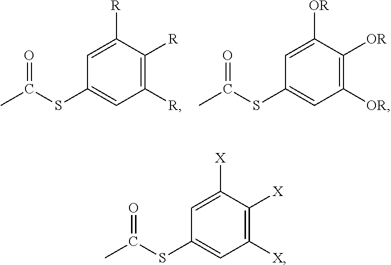

### —C(═O)—NRR,

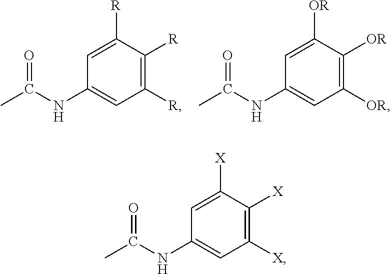

### —C(═O)OM, —S(═O)OM, —S(═O)(═O)OM,

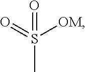

### —NO₂, —OR, —OC(═O)R,

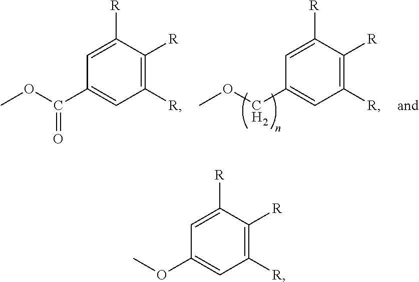

### —H, —F, —Cl, and —CH₃,

wherein each R is independently H, —NO₂, a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein X is a halogen or hydrogen, at least one X is a halogen, M is an organic or inorganic cation, and n=0 to 20 and
wherein Formula III is

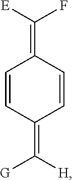

wherein each E, F, G, and H is independently —C(C═O)OR or —CN, wherein each R is independently a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein the monomers are chemically bound to each other by at least one carbon-carbon single bond to form the crystalline polymer;
mixing the crystalline polymer with a solvent to form a mixture;
sonicating the mixture to form a suspension; and
vacuum filtering and heat pressing the suspension. The carbon-carbon single bond can have a bond length between about 1.57 Å and about 1.70 Å. The polymer can have a depolymerization temperature between about 125° C. to about 375° C. The solvent can be an organic solvent. The solvent can be acetone. The solvent can be chloroform. Ultrasound can be performed at an amplitude of about 20, about 4 second pulse and about 1 second rest. Ultrasound can be performed at an amplitude of about 15, about 4 second pulse and about 1 second rest. Ultrasound can be performed at a temperature below 0° C. A and B can be

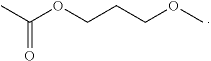

The organic cation can be

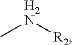

wherein R₂ is H, —CH₂-naphtyl, —CH₂-phenyl, methyl, ethyl, n-propyl, iso-propyl, butyl, n-butyl, sec-butyl, or iso-butyl.

Also provided is a thermoplastic production process comprising:
providing a crystalline polymer formed from at least two monomers independently selected from the group consisting of Formula I, Formula II, and Formula III,
wherein Formula I is

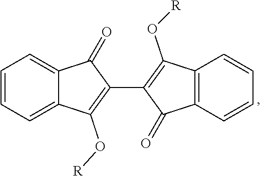

wherein each R is independently C₁ to C₂₀ linear chain alkyl or a C₁ to C₂₀ branched chain alkyl, wherein any —H may be substituted with —F,
wherein Formula II is

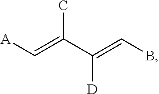

wherein A, B, C and D are independently selected from —C(═O)—R, —C(═O)—OR,

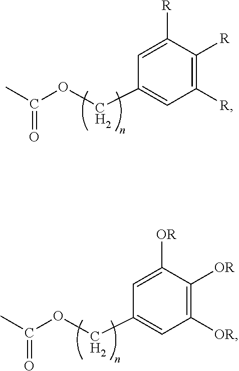

### —C(═S)—R,

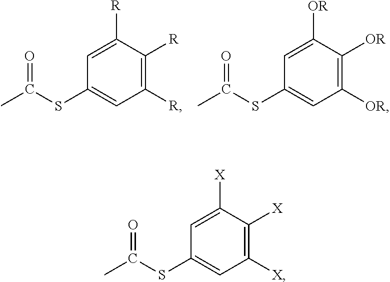

### —C(═O)—NRR,

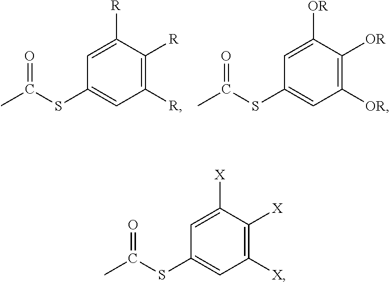

### —C(═O)OM, —S(═O)OM, —S(═O)(═O)OM,

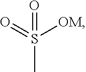

### —NO₂, —OR, —OC(═O)R,

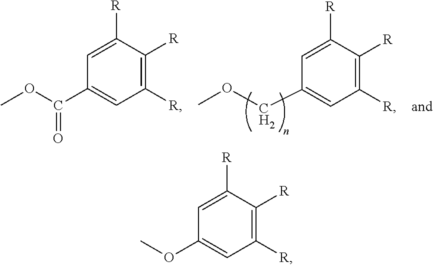

### —H, —F, —Cl, and —CH₃,

wherein each R is independently H, —NO₂, a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein X is a halogen or hydrogen, at least one X is a halogen, M is an organic or inorganic cation, and n=0 to 20, and
wherein Formula III is

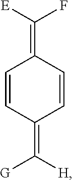

wherein each E, F, G, and H is independently —C(C═O)OR or —CN, wherein each R is independently a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein the monomers are chemically bound to each other by at least one carbon-carbon single bond to form the crystalline polymer;
mixing the crystalline polymer with a first solvent to form a mixture;
subjecting the mixture to heat to form a viscous polymer solution;
mixing the viscous polymer solution with a second solvent to form a residue; and
drying the reside. The carbon-carbon single bond can have a bond length between about 1.57 Å and about 1.70 Å. The polymer can have a depolymerization temperature between about 125° C. to about 375° C. A and B are

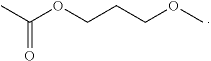

Further provided is a method of depolymerization comprising:
providing a crystalline polymer formed from at least two monomers independently selected from the group consisting of Formula I, Formula II, and Formula III,
wherein Formula I is

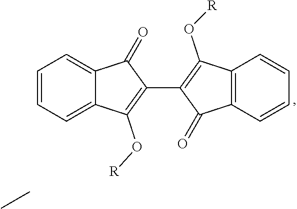

wherein each R is independently a C₁ to C₂₀ linear chain alkyl or a C₁ to C₂₀ branched chain alkyl, wherein any —H maybe substituted with —F,
wherein Formula II is

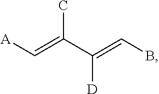

wherein A, B, C and D are independently selected from —C(═O)—R, —C(═O)—OR,

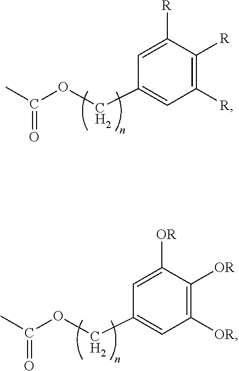

### —C(═S)R,

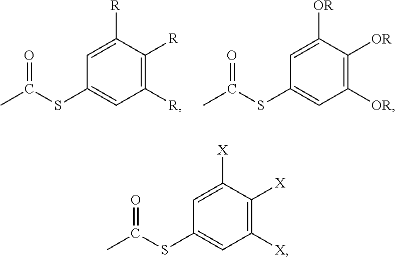

### —C(═O)—NRR,

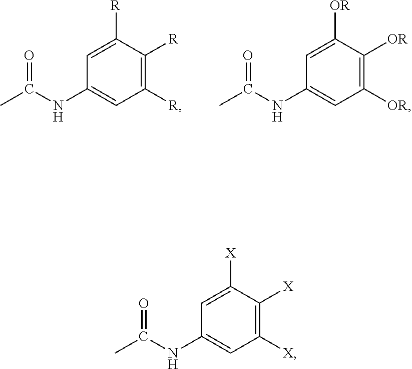

### —C(═O)OM, —S(═O)OM, —S(═O)(═O)OM,

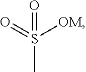

### —NO₂, —OR, —OC(═O)R,

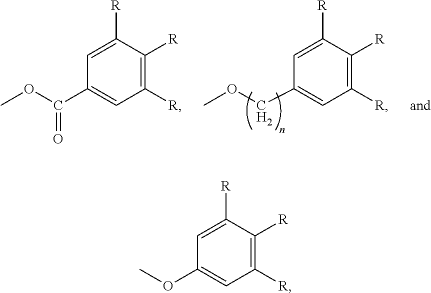

### —H, —F, —Cl, and —CH₃,

wherein each R is independently H, —NO₂, a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein X is a halogen or hydrogen, at least one X is a halogen, M is an organic or inorganic cation, and n=0 to 20, and
wherein Formula III is

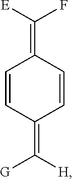

wherein each E, F, G, and H is independently —C(C═O)OR or —CN, wherein each R is independently a C₁ to C₂₀ branched chain alkyl or a C₁ to C₂₀ straight chain alkyl, optionally substituted with —O—, —NRR or —S—, and any hydrogen can be substituted with —F,
wherein the monomers are chemically bound to each other by at least one carbon-carbon single bond to form the crystalline polymer,
wherein the carbon-carbon single bond has a bond length between about 1.57 Å and about 1.70 Å,
mixing the crystalline polymer with a solvent to form a mixture,
heating the mixture to a temperature between about 125° C. to about 375° C., and
recovering the monomers. The temperature can be between about 240° C. and about 275° C. or between about 310° C. and about 355° C. A and B can be

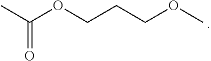

The organic cation can be

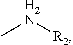

wherein R₂ is H, —CH₂-naphtyl, —CH₂-phenyl, methyl, ethyl, n-propyl, iso-propyl, butyl, n-butyl, sec-butyl, or iso-butyl. The carbon-carbon single bond can have a bond length between about 1.57 Å and about 1.70 Å between about 1.59 Å and
about 1.67 Å or between about 1.61 Å and about 1.63 Å.

### EXAMPLES

The following examples serve to illustrate the present disclosure and are not intended to limit its scope in any way.

### Example 1

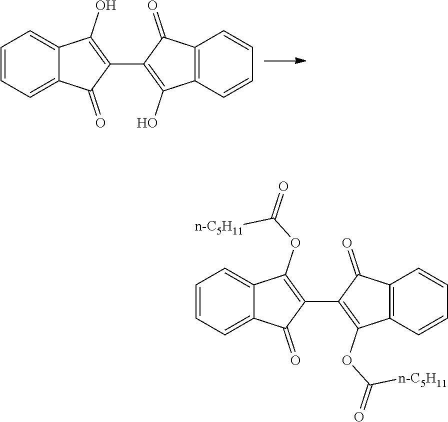

To a two-neck round-bottom flask, [2,2′-bi-1H-indene]-3,3′-dihydroxy-1,1′-dione (BIT-OH, 0.50 g, 1.7 mmol) was added in dry chloroform (30 mL) under argon atmosphere. The mixture was cooled to −15° C. in a salt ice bath, and 1.4 mL triethylamine were added. BIT-OH dissolved, and a purple solution formed. Hexanoyl chloride (0.95 mL, 6.8 mmol) was added dropwise to the solution at −15° C. The solution gradually turned orange during the 2 hours reaction at −15° C. and was then quenched by water. After being washed with brine and dried with MgSO₄, the crude product was purified by silica column chromatography (CH₂Cl₂ as eluent). By adding 10 mL ethanol to its 2 mL DCM solution, pure BIT-6 precipitated as orange needle-like crystals (0.61 g, 74% yield). ¹H NMR (400 MHz, CDCl₃, ppm) δ: 7.43 (d, J=7.1 Hz, C₆H₄, 2H), 7.37 (t, J=7.5 Hz, C₆H₄, 2H), 7.27 (t, J=7.9 Hz, C₆H₄, 2H), 7.10 (d, J=7.2 Hz, C₆H₄, 2H), 2.65 (t, J=7.4 Hz, CH₂COOR, 4H), 1.88-1.66 (m, CH₂CH₂COOR, 4H), 1.43-1.28 (m, CH₂, 8H), 0.90 (t, J=7.1 Hz, CH₃, 6H). ¹³C NMR (100 MHz, CDCl₃, ppm) δ:192.26, 169.09, 164.26, 140.27, 133.26, 130.74, 129.79, 122.18, 119.78, 112.46, 34.03, 31.02, 24.04, 22.22, 13.80. HRMS (ESI) calculated for C₃₀H₃₀O₆Na⁺ ([M+Na]⁺): 509.1935; found: 509.1930.

### Example 2

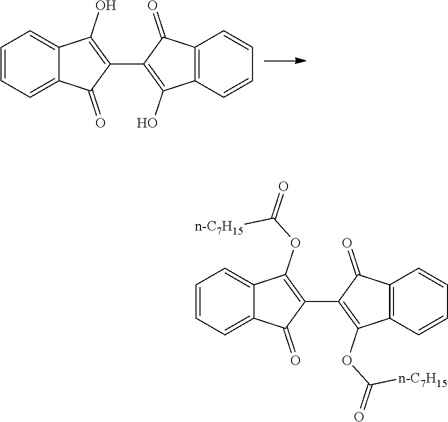

To a two-neck round-bottom flask, [2,2′-bi-1H-indene]-3,3′-dihydroxy-1,1′-dione (BIT-OH, 0.50 g, 1.7 mmol) was added in dry chloroform (30 mL) under argon atmosphere. The mixture was cooled to −15° C. in a salt ice bath, and 1.4 mL triethylamine were added. BIT-OH dissolved, and a purple solution formed. Octanoyl chloride (1.17 mL, 6.8 mmol) was added dropwise to the solution at −15° C. The solution gradually turned orange during the 2 hours reaction at −15° C. and was then quenched by water. After being washed with brine and dried with MgSO₄, the crude product was purified by silica column chromatography (CH₂Cl₂ as eluent). By adding 10 mL ethanol to its 2 mL DCM solution, pure BIT-8 precipitated as orange needle-like crystals (0.63 g, 75% yield). ¹H NMR (400 MHz, CDCl₃, ppm) δ: 7.43 (d, J=7.1 Hz, C₆H₄, 2H), 7.37 (t, J=7.5 Hz, C₆H₄, 2H), 7.27 (t, J=7.4 Hz, C₆H₄, 2H), 7.10 (d, J=7.2 Hz, C₆H₄, 2H), 2.65 (t, J=7.4 Hz, CH₂COOR, 4H), 1.72 (p, J=7.5 Hz, CH₂CH₂COOR, 4H), 1.46-1.20 (m, CH₂, 16H), 0.88 (t, J=6.9 Hz, CH₃, 6H). ¹³C NMR (100 MHz, CDCl₃, ppm) δ:192.25, 169.10, 164.29, 140.26, 133.25, 130.74, 129.78, 122.18, 119.78, 112.47, 34.08, 31.55, 28.85, 28.83, 24.36, 22.49, 13.97. HRMS (ESI) calculated for C₃₄H₃₈O₆Na⁺ ([M+Na]⁺): 565.2561; found: 565.2555.

### Example 3

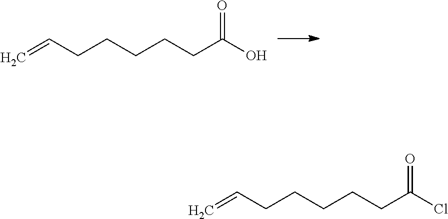

In a two-neck round-bottom flask, oxalyl chloride (2.32 g, 18.28 mmol) was dissolved in 4 mL of anhydrous chloroform. Oct-enoic acid (2.00 g, 14.06 mmol) was dissolved in 4 mL of anhydrous chloroform and was added dropwise to the previous solution. After the addition, five drops of DMF were added, and the mixture was stirred at room temperature for 30 minutes. Then Et₂O was added, and the mixture was filtered and concentrated under reduced pressure to give oct-enoyl chloride (2.146 g, 95% yield). H NMR (400 MHz, CDCl₃) δ 5.77 (ddt, J=16.8, 10.2, 6.7 Hz, CH, 1H), 5.05-4.86 (m, CH₂, 2H), 2.87 (td, J=7.3, 1.5 Hz, CH₂, 2H), 2.04 (q, J=7.0 Hz, CH₂, 2H), 1.79-1.62 (m, CH₂, 2H), 1.37 (qq, J=9.0, 4.4 Hz, CH₂, 4H).

### Example 4

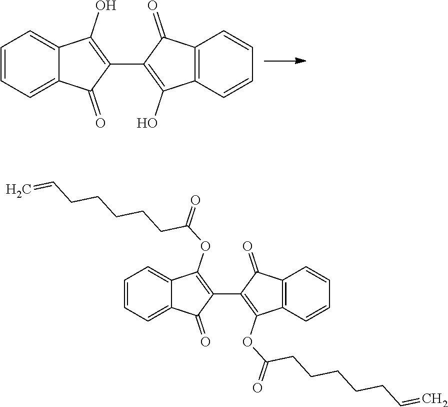 [SMILES: O=C1C(C2=C(O)c3ccccc3C2=O)=C(O)c2ccccc21 ; C=CCCCCCC(=O)OC1=C(C2=C(OC(=O)CCCCCC=C)c3ccccc3C2=O)C(=O)c2ccccc21]

To a two-neck round-bottom flask, [2,2′-bi-1H-indene]-3,3′-dihydroxy-1,1′-dione (BIT-OH, 0.76 g, 2.59 mmol) was added in dry chloroform (30 mL) under argon atmosphere. The mixture was cooled to −15° C. in a salt ice bath, and 0.92 mL N,N-Diisopropylethylamine was added. BIT-OH dissolved, and a purple solution formed. Oct-7-enoyl chloride (1.04 g, 6.47 mmol) was added dropwise to the solution at −15° C. The solution gradually turned orange during the 2 hours reaction at −15° C. and was then quenched by water. After being washed with brine and dried with MgSO₄, the crude product was purified by silica column chromatography (CH₂Cl₂ as eluent). By adding 10 mL ethanol to its 2 mL DCM solution, pure BIT-8D precipitated as orange needle-like crystals (1.18 g, 85% yield). ¹H NMR (400 MHz, CDCl₃) δ 7.42 (dd, J=6.9, 0.9 Hz, C₆H₄, 1H), 7.37 (td, J=7.8, 1.4 Hz, C₆H₄, 1H), 7.28 (d, J=7.3 Hz, C₆H₄, 1H), 7.10 (d, J=6.9 Hz, C₆H₄, 1H), 5.79 (ddt, J=16.9, 10.2, 6.7 Hz, CH, 1H), 5.04-4.90 (m, CH₂, 2H), 2.65 (t, J=7.4 Hz, CH₂COOR, 2H), 2.05 (dtd, J=7.3, 5.5, 2.7 Hz, CH₂CH₂COOR, 2H), 1.73 (tdd, J=7.4, 6.0, 3.2 Hz, CH₂, 2H), 1.49-1.36 (m, CH₂, 4H). ¹³C NMR (101 MHz, CDCl₃) δ 192.30, 169.05, 164.25, 140.26, 138.63, 133.29, 130.73, 129.82, 122.21, 119.81, 114.41, 112.46, 34.00, 33.43, 28.41, 28.33, 24.19. HRMS (ESI) calculated for C₃₄H₃₄O₆Na⁺ ([M+Na]⁺): 561.2248; found: 561.2242.

### Example 5

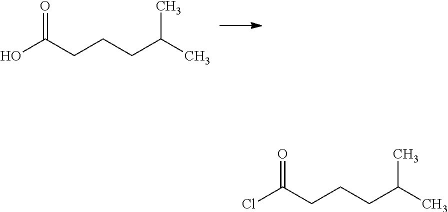

In a two-neck round-bottom flask, thionyl chloride (1.20 mL, 16.30 mmol) was added under argon atmosphere. 5-methylhexanoic acid (2.02 g, 15.54 mmol) was added dropwise to the flask. The mixture was heated under reflux for 2 h. Then the mixture was cooled to room temperature, and extra thionyl chloride was removed under reduced pressure to give a colorless liquid (2.26 g, 98% yield). ¹H NMR (400 MHz, CDCl₃) δ 2.84 (t, J=7.2 Hz, CH₂, 2H), 1.75-1.67 (m, CH₂, 1H), 1.66 (t, J=7.4 Hz, CH₂, 1H), 1.54 (hept, J=7.1 Hz, CH, 1H), 1.21 (q, J=7.5 Hz, CH₂, 2H), 0.90-0.84 (m, CH₃, 6H).

### Example 6

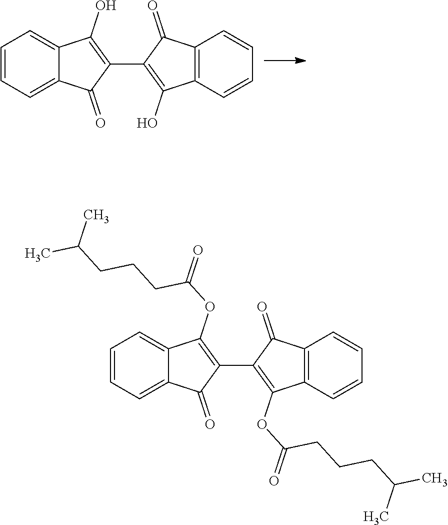 [SMILES: O=C1C(C2=C(O)c3ccccc3C2=O)=C(O)c2ccccc21 ; CC(C)CCCC(=O)OC1=C(C2=C(OC(=O)CCCC(C)C)c3ccccc3C2=O)C(=O)c2ccccc21]

To a two-neck round-bottom flask, [2,2′-bi-1H-indene]-3,3′-dihydroxy-1,1′-dione (BIT-OH, 0.40 g, 1.37 mmol) was added in dry chloroform (20 mL) under argon atmosphere. The mixture was cooled to −15° C. in a salt ice bath, and 0.52 mL N,N-diisopropylethylamine was added. BIT-OH dissolved, and a purple solution formed. 5-methylhexanoyl chloride (0.51 g, 3.49 mmol) was added dropwise to the solution at −15° C. The solution gradually turned orange during the 2 hours reaction at −15° C. and was then quenched by water. After being washed with brine and dried with MgSO₄, the crude product was purified by silica column chromatography (CH₂Cl₂ as eluent). By adding 10 mL ethanol to its 2 mL DCM solution, pure BIT-7B precipitated as orange needle-like crystals (0.56 g, 80% yield). ¹H NMR (400 MHz, CDCl₃) δ 7.46-7.33 (m, C₆H₄, 2H), 7.31-7.22 (m, C₆H₄, 1H), 7.10 (d, J=7.2 Hz, C₆H₄, 1H), 2.63 (t, J=7.5 Hz, CH₂COOR, 2H), 1.79-1.66 (m, CH₂CH₂COOR, 2H), 1.66-1.49 (m, J=6.4 Hz, CH, 1H), 1.33-1.22 (m, CH₂, 2H), 0.89 (d, J=6.6 Hz, CH₃, 6H). ¹³C NMR (101 MHz, CDCl₃) δ 192.28, 169.11, 164.28, 140.28, 133.28, 130.75, 129.80, 122.21, 119.80, 112.46, 38.03, 34.28, 27.67, 22.36, 22.26. HRMS (ESI) calculated for C₃₂H₃₄O₆Na⁺ ([M+Na]⁺): 537.2248; found: 537.2241.

### Example 7

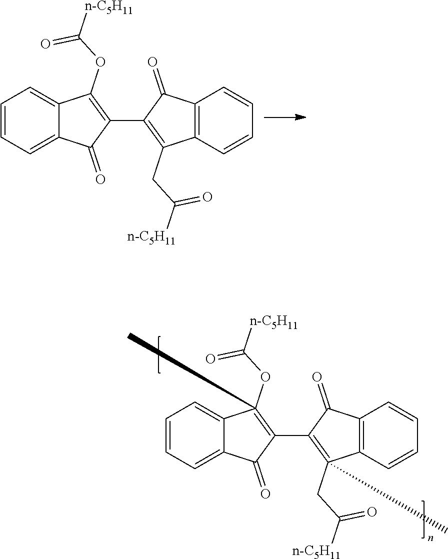

BIT-6 crystals (100 mg) were placed in a glass petri dish and covered with a glass lid. The monomer was irradiated under visible-light lamps in the photoreactor for 12 h. The resulting polymer crystals were stirred in 50 mL chloroform at reflux for 12 h to remove any unreacted monomer/oligomer. Filtration provided the polymer polyBIT-6 as light yellow needle-like crystals (99.5 mg, >99% yield). The polymer is insoluble in common solvents. The polymer structure was confirmed by single crystal X-ray crystallography.

### Example 8

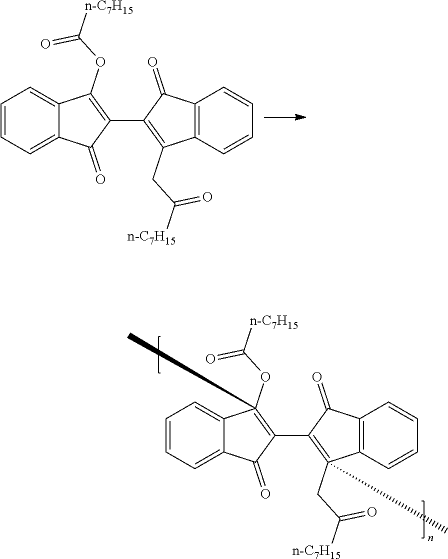

BIT-8 crystals (100 mg) were placed in a glass petri dish and covered with a glass lid. The monomer was irradiated under visible-light lamps in the photoreactor for 12 h. The resulting polymer crystals were stirred in 50 mL chloroform at reflux for 12 h to remove any unreacted monomer/oligomer. Filtration provided the polymer polyBIT-8 as light yellow needle-like crystals (99.5 mg, >99% yield). The polymer is insoluble in common solvents. The polymer structure was confirmed by single crystal X-ray crystallography.

### Example 9

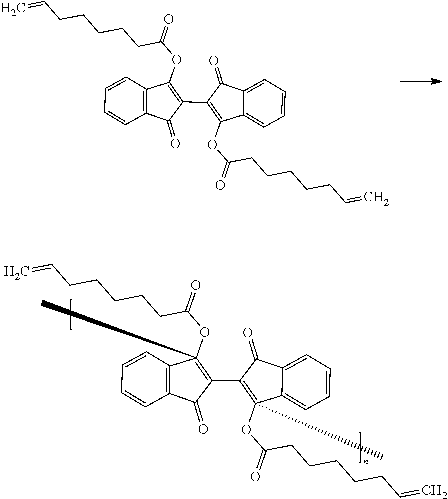

BIT-8D crystals (100 mg) were placed in a glass petri dish and covered with a glass lid. The monomer was irradiated under visible-light lamps in the photoreactor for 72 h. The resulting polymer crystals were stirred in 50 mL chloroform at reflux for 12 h to remove any unreacted monomer/oligomer. Filtration provided the polymer polyBIT-8D as light yellow needle-like crystals (99.5 mg, >99% yield). The polymer is insoluble in common solvents. The polymer structure was confirmed by single crystal X-ray crystallography.

### Example 10

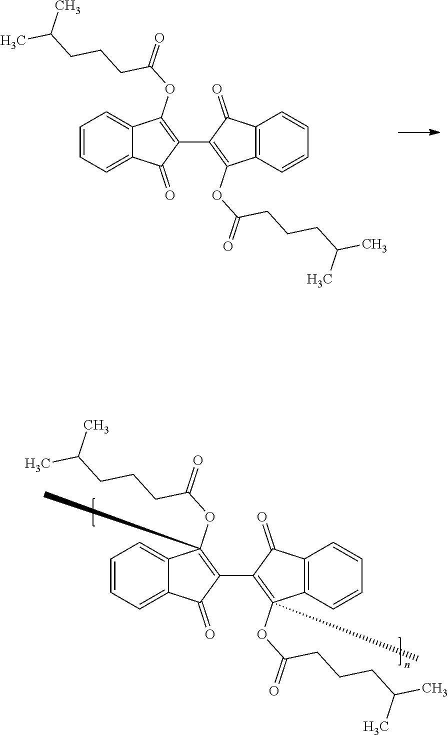

BIT-7B crystals (100 mg) were placed in a glass petri dish and covered with a glass lid. The monomer was irradiated under visible-light lamps in the photoreactor for 12 h. The resulting polymer crystals were stirred in 50 mL chloroform at reflux for 12 h to remove any unreacted monomer/oligomer. Filtration provided the polymer polyBIT-7B as light yellow needle-like crystals (99.5 mg, >99% yield). The polymer is insoluble in common solvents. The polymer structure was confirmed by single crystal X-ray crystallography.

### Example 11

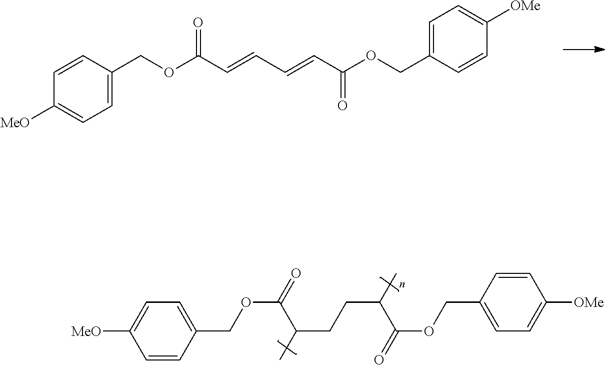

(E,E)-ME-OMeB monomer crystals (500 mg) were placed in a glass petri dish and covered with a glass lid. The monomer was irradiated under UVA lamps (350 nm) in the photoreactor for 72 h. The resulting polymer crystals were stirred in 50 mL chloroform at reflux for 12 h to remove any unreacted monomer/oligomer. Filtration provided the polymer polyME-OMeB as white plate-like crystals (492 mg, 98% yield). The polymer structure was confirmed by single crystal X-ray crystallography. The polymer crystals were melted at −205° C. and formed a colorless solid after cooling down. This polymer form dissolved in chloroform and was analyzed by ¹H-NMR. H NMR (400 MHz, CDCl₃, ppm) δ: 7.12 (d, J=8.7 Hz, C₆H₄, 4H), 6.75 (d, J=8.6 Hz, C₆H₄, 4H), 5.35 (s, CH═, 2H), 4.90-4.77 (m, OCH₂, 4H), 3.67 (s, OCH₃, 6H), 3.39 (s, CH, 2H).

### Example 12

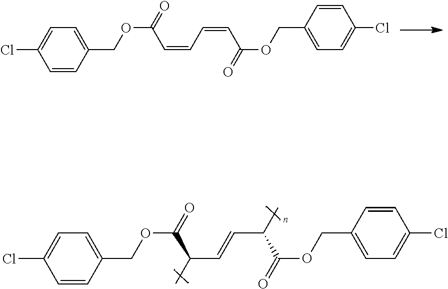

(Z,Z)-ME-CB monomer crystals (500 mg) were placed in a glass petri dish and covered with a glass lid. The monomer was irradiated under UVA lamps (350 nm) in the photoreactor for 48 h. The resulting polymer crystals were stirred in 50 mL chloroform at reflux for 12 h to remove any unreacted monomer/oligomer. Filtration gave the polymer polyME-CB as white needle-like crystals (497 mg, 99% yield). The polymer is insoluble in common solvents. The polymer structure was confirmed by single crystal X-ray crystallography.

### Example 13

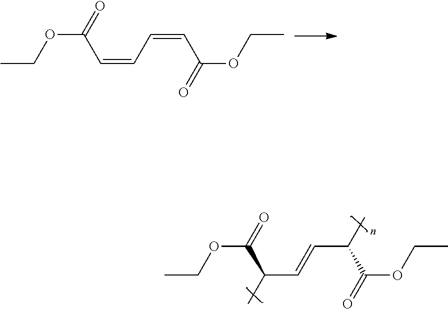

(Z,Z)-ME-Et monomer crystals (500 mg) were placed in a glass petri dish and covered with a glass lid. The monomer was irradiated under UVA lamps (350 nm) in the photoreactor for 48 h. The resulting polymer crystals were stirred in 50 mL chloroform at reflux for 12 h to remove any unreacted monomer/oligomer. Filtration gave the polymer polyME-Et as white needle-like crystals (482 mg, 96% yield). The polymer is insoluble in common solvents. The polymer structure was confirmed by single crystal X-ray crystallography.

### Example 14

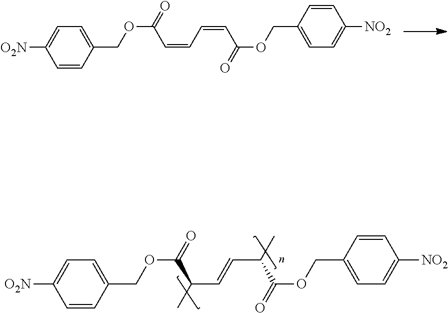

(Z,Z)-ME-NB monomer crystals (500 mg) were placed in a glass petri dish and covered with a glass lid. The monomer was irradiated under UVA lamps (350 nm) in the photoreactor for 48 h. The resulting polymer crystals were stirred in 50 mL chloroform at reflux for 12 h to remove any unreacted monomer/oligomer. Filtration gave the polymer polyME-NB as pale-yellow needle-like crystals (488 mg, 98% yield). The polymer is insoluble in common solvents. The polymer structure was confirmed by single crystal X-ray crystallography.

### Example 15

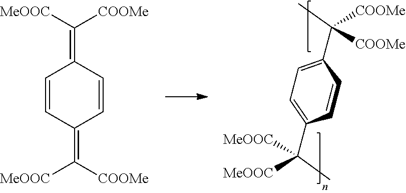

QDM-Me monomer crystals (100 mg) were placed in a glass petri dish and covered with a glass lid. The monomer was irradiated under UVA lamps (350 nm) in N2 atmosphere for 12 h. The resulting polymer crystals were stirred in 50 mL chloroform at reflux for 12 h to remove any unreacted monomer/oligomer. The yellow polymer crystals were dispersed into smaller particles during this chloroform purification process. Filtration gave the polymer polyQDM-Me as an off-white powder (87 mg, 87% yield). The polymer is insoluble in common solvents. The polymer structure was confirmed by single crystal X-ray crystallography using crude yellow polymer crystals.

### Example 16

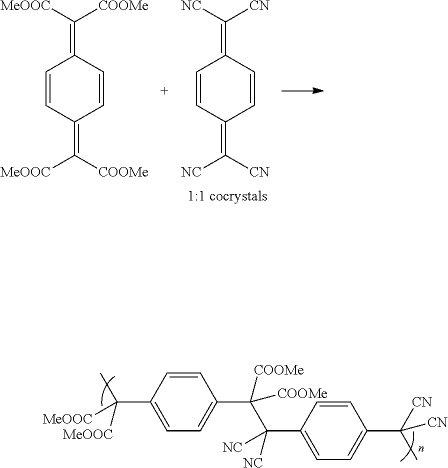

QDM-TCNQ monomer cocrystals (250 mg) were placed in a glass petri dish and covered with a glass lid. The monomer was irradiated under UVA lamps (350 nm) in N₂ atmosphere for 12 h. The resulting polymer crystals were stirred in 50 mL chloroform at reflux for 12 h to remove any unreacted monomer/oligomer. The yellow polymer crystals were dispersed into smaller particles during this chloroform purification process. Filtration gave the polymer polyQDM-TCNQ as an ivory color prism-like crystals (212 mg, 85% yield). The polymer is insoluble in common solvents. The polymer structure was confirmed by single crystal X-ray crystallography using crude yellow polymer crystals.

### Example 17

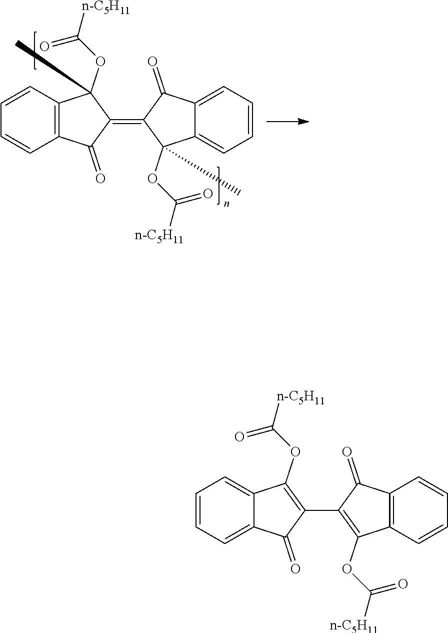

In a 25 mL round-bottom flask with a condenser, polyBIT-6 crystals (50.3 mg) were added to 3 mL anisole under argon atmosphere. The mixture was heated at 145° C. for 30 minutes. Polymer crystals dissolved, and the solution color changed from colorless to orange. After cooling down, anisole was removed from the resulting mixture by a short silica column (CH₂Cl₂ as eluent). 49.8 mg of BIT-6 was obtained after evaporation as an orange powder (99% yield).

### Example 18

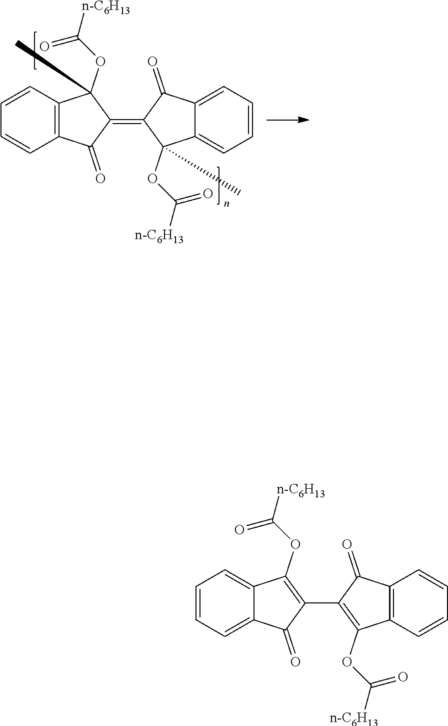

In a 25 mL round-bottom flask with a condenser, polyBIT-7 crystals (58.6 mg) were added to 3 mL anisole under argon atmosphere. The mixture was heated at 145° C. for 30 minutes. Polymer crystals dissolved, and the solution color changed from colorless to orange. After cooling down, anisole was removed from the resulting mixture by a short silica column (CH₂Cl₂ as eluent). 57.8 mg of BIT-7 was obtained after evaporation as an orange powder (99% yield).

### Example 19

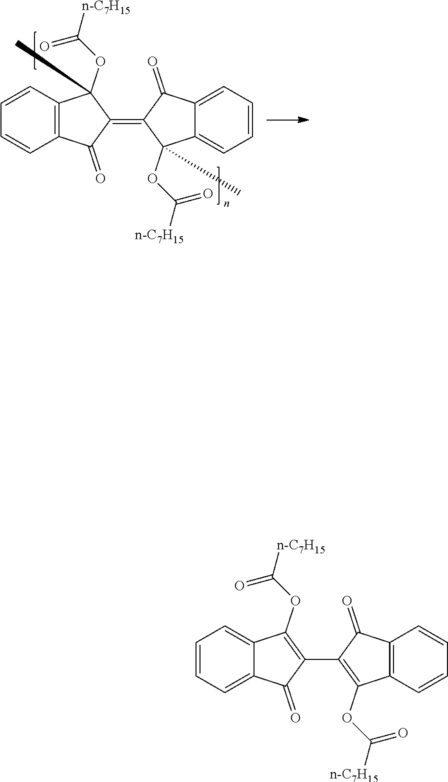

In a 25 mL round-bottom flask with a condenser, polyBIT-8 crystals (99.6 mg) were added to 5 mL anisole under argon atmosphere. The mixture was heated at 145° C. for 30 minutes. Polymer crystals dissolved, and the solution color changed from colorless to orange. After cooling down, anisole was removed from the resulting mixture by a short silica column (CH₂Cl₂ as eluent). 99.5 mg of BIT-8 was obtained after evaporation as an orange powder (>99% yield). The depolymerization reaction of the pristine thin film (39.4 mg) was conducted with the same method as polyBIT-8 crystals. 31.8 mg of BIT-8 were obtained (98% yield).

### Example 20

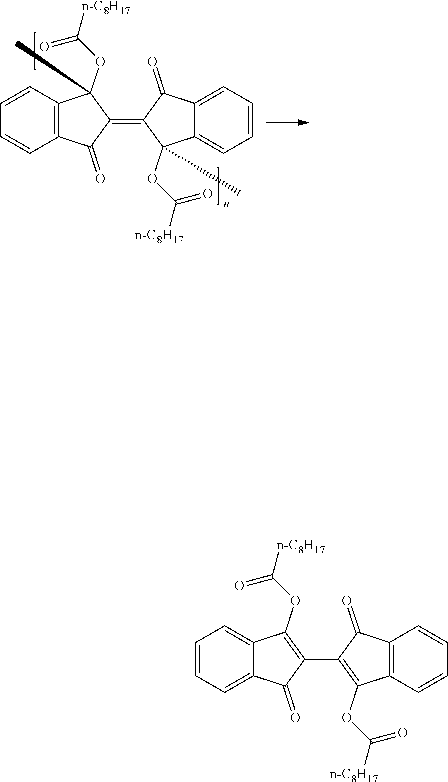

In a 25 mL round-bottom flask with a condenser, polyBIT-9 crystals (47.8 mg) were added to 5 mL anisole under argon atmosphere. The mixture was heated at 145° C. for 30 minutes. Polymer crystals dissolved, and the solution color changed from colorless to orange. After cooling down, anisole was removed from the resulting mixture by a short silica column (CH₂Cl₂ as eluent). 47.3 mg of BIT-9 was obtained after evaporation as an orange powder (99% yield).

### Example 21

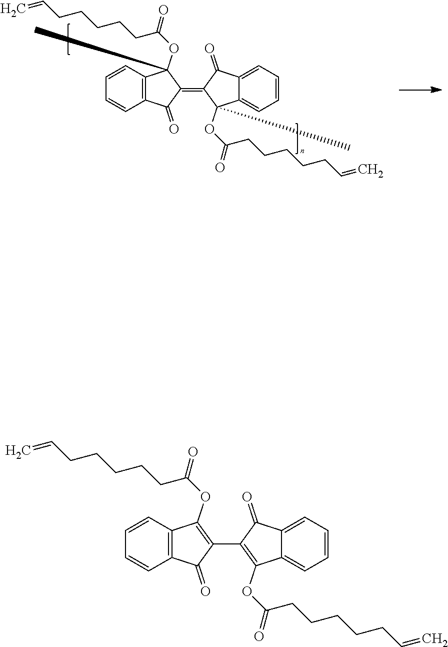

In a 25 mL round-bottom flask with a condenser, polyBIT-8D crystals (40.5 mg) were added to 3 mL anisole under argon atmosphere. The mixture was heated at 155° C. for 1 h. Polymer crystals dissolved, and the solution color changed from colorless to orange. After cooling down, anisole was removed from the resulting mixture by a short silica column (CH₂Cl₂ as eluent). 39.9 mg of BIT-8D was obtained after evaporation as an orange powder (98.5% yield).

### Example 22

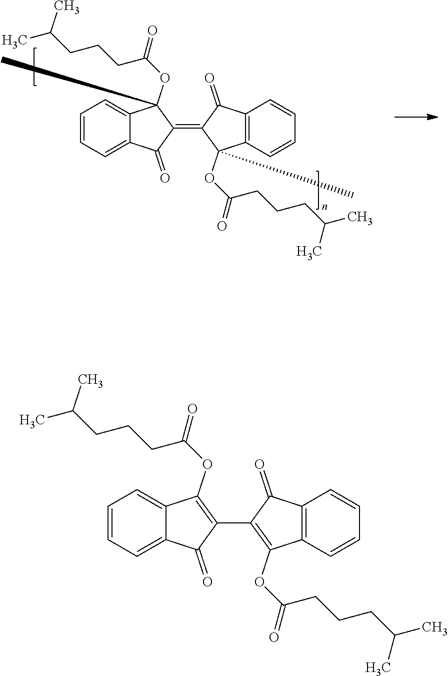

In a 25 mL round-bottom flask with a condenser, polyBIT-7B crystals (45.0 mg) were added to 3 mL anisole under argon atmosphere. The mixture was heated at 135° C. for 30 minutes. Polymer crystals dissolved, and the solution color changed from colorless to orange. After cooling down, anisole was removed from the resulting mixture by a short silica column (CH₂Cl₂ as eluent). 44.5 mg of BIT-7B was obtained after evaporation as an orange powder (99% yield).

### Example 23

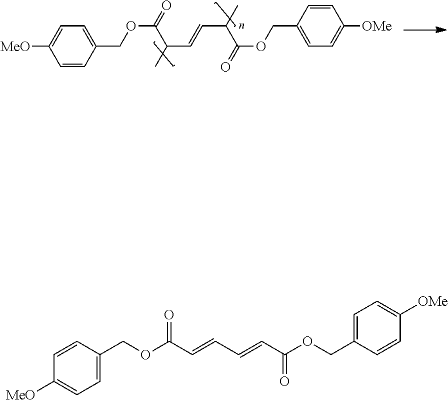

polyME-OMeB crystals (50.2 mg) were mixed with diphenyl ether (20 mL) in a 50 mL round-bottom flask with a condenser under argon atmosphere. The reaction mixture was heated at reflux (˜258° C.) for 20 min. The reaction setup was cooled down quickly by water (to prevent from possible thermal polymerization-depolymerization equilibrium). The solvent diphenyl ether was removed by a short silica column (CH₂Cl₂:Ethyl Acetate=15:1) to obtain crude ME-CB with EE (major), EZ and ZZ isomers (86% crude yield, all isomers are polymerizable). The I2 treatment was applied as a catalyst to facilitate thermal equilibrium reaction of EE/EZ/ZZ isomers. Crude ME-OMeB isomers were mixed with 0.6 mg I2 (0.02 eq) in 5 mL THF, stirred at room temperature under argon atmosphere overnight, then evaporated under vacuum. The residue was purified by silica column chromatography (CH₂C₂) to give 37.2 mg of pure (EE)-ME-OMeB as a white powder (41.0 mg, 82% yield). The EZ isomer was too little to be collected. ¹H NMR (400 MHz, CDCl₃, ppm) δ: 7.34-7.28 (m, CH═CHCO₂R and C₆H₄, 6H), 6.92-6.87 (m, C₆H₄, 4H), 6.25-6.14 (m, CH═CHCO₂R, 2H), 5.14 (s, OCH₂, 4H), 3.81 (s, CH₃, 6H).

The depolymerization also occurred at lower temperature in a boiling 2:1 solvent mixture of diphenyl ether and trichlorobenzene (−228° C.), but with longer reaction time of 3 h, affording 60% recovery yield (60 mg) of ME-OMeB monomers.

The depolymerization of the sonication-processed film before heat-pressing and after heat pressing was conducted with the same method as polyME-OMeB crystals with recovery yields of 83% and 73%, respectively. The depolymerization reaction of the hot-solvent processed polymer (50.0 mg) was conducted with the same method as polyME-OMeB crystals. 35.9 mg of ME-OMeB was obtained (72% yield).

### Example 24

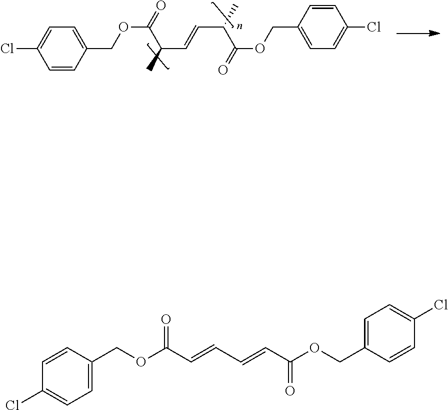

polyME-CB crystals (100 mg) were mixed with diphenyl ether (15 mL) in a 50 mL round-bottom flask with a condenser under argon atmosphere. The reaction mixture was heated at reflux (˜258° C.) for 20 min. The reaction setup was cooled down quickly by water (to prevent from possible thermal polymerization-depolymerization equilibrium). The solvent diphenyl ether was removed by a short silica column (CH₂Cl₂:Ethyl Acetate=15:1) to obtain crude ME-CB with EE (major), EZ and ZZ isomers (95% crude yield). Crude ME-CB isomers were mixed with 1.3 mg I2 (0.02 eq) in 5 mL THF, stirred at 60° C. under argon atmosphere overnight, then evaporated under vacuum. The residue was purified by silica column chromatography (CH₂C₂) to give 85 mg of pure (EE)-ME-CB. and 6 mg of (EZ)-ME-CB as white powders (91% yield in total). (EE)-ME-CB: ¹H NMR (400 MHz, CDCl₃, ppm) δ: 7.38-7.28 (m, CH═CHCO₂R and C₆H₄, 10H), 6.28-6.18 (m, CH═CHCO₂R, 2H), 5.17 (s, OCH₂, 4H). (EZ)-ME-CB: ¹H NMR (400 MHz, CDCl₃, ppm) δ: 8.45 (ddd, J=15.6, 11.6, 1.1 Hz, CH═CHCO₂R, 1H), 7.37-7.29 (m, C₆H₄, 8H), 6.67 (t, J=11.6 Hz, CH═CHCO₂R, 1H), 6.14 (d, J=15.6 Hz, CH═CHCO₂R, 1H), 6.00 (d, J=11.4 Hz, CH═CHCO₂R, 1H), 5.18 (s, OCH₂, 2H), 5.16 (s, OCH₂, 2H).

The depolymerization reaction also occurs at lower temperature in a boiling 2:1 solvent mixture of diphenyl ether and trichlorobenzene (˜228° C.), but with longer reaction time of 2.5 h, affording 81% recovery yield (80.9 mg) of ME-CB monomers.

The depolymerization of the sonication-processed polyME-CB film before heat-pressing and after heat pressing was conducted with the same method as polyME-OMeB crystals with recovery yields of 93% and 92%, respectively.

### Example 25

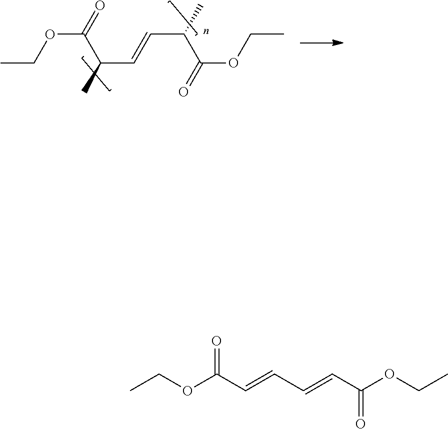

polyME-Et crystals (100 mg) were mixed with diphenyl ether (15 mL) in a 50 mL round-bottom flask with a condenser under argon atmosphere. The reaction mixture was heated at reflux (˜258° C.) for 20 min. The reaction setup was cooled down quickly by water (to prevent from possible thermal polymerization-depolymerization equilibrium). The solvent diphenyl ether was removed by a short silica column (CH₂C₂) to obtain crude ME-Et with EE (major), EZ and ZZ isomers (91% crude yield). Crude ME-CB isomers were mixed with 1.3 mg I2 (0.02 eq) in 5 mL THF, stirred at 60° C. under argon atmosphere overnight, then evaporated under vacuum. The residue was purified by silica column chromatography (CH₂C₂) to give 84 mg of pure (EE)-ME-Et as white powders (84% yield in total). (EE)-ME-Et: ¹H NMR (400 MHz, CDCl₃, ppm) δ: 7.38-7.28 (m, CH═CHCO₂R and C₆H₄, 10H), 6.28-6.18 (m, CH═CHCO₂R, 2H), 5.17 (s, OCH₂, 4H). (EZ)-ME-CB: ¹H NMR (400 MHz, CDCl₃, ppm) δ: 7.48-7.16 (m, CH═CHCO₂R, 2H), 6.28-6.11 (m, CH═CHCO₂R, 2H), 4.23 (q, OCH₂, J=7.1 Hz, 4H), 1.31 (t, CH₃, J=7.1 Hz, 6H).

### Example 26

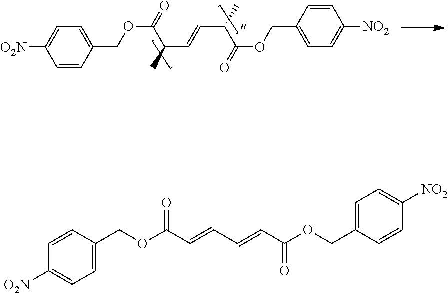

polyME-NB crystals (100 mg) were mixed with diphenyl ether (15 mL) and trichlorobenzene (3 mL) in a 50 mL round-bottom flask with a condenser under argon atmosphere. The reaction mixture was heated at reflux (−258° C.) for 20 min. The reaction setup was cooled down quickly by water (to prevent from possible thermal polymerization-depolymerization equilibrium). The solvent diphenyl ether was removed by a short silica column (CH₂Cl₂:Ethyl Acetate=15:1) to obtain crude ME-NB with EE (major), EZ and ZZ isomers (26% crude yield). Crude ME-CB isomers were mixed with 1.3 mg I2 (0.02 eq) in 5 mL THF, stirred at 60° C. under argon atmosphere overnight, then evaporated under vacuum. The residue was purified by silica column chromatography (CH₂C₂) to give 22 mg of pure (EE)-ME-Et as white powders (22% yield in total). (EE)-ME-NB: ¹H NMR (400 MHz, CDCl₃, ppm) δ: 8.24 (d, J=8.7 Hz, C₆H₄, 2H), 7.54 (d, J=8.9 Hz, C₆H₄, 2H), 7.45-7.33 (m, CH═CHCO₂R, 2H), 6.35-6.24 (m, CH═CHCO₂R, 2H), 5.31 (s, OCH₂, 4H).

### Example 27

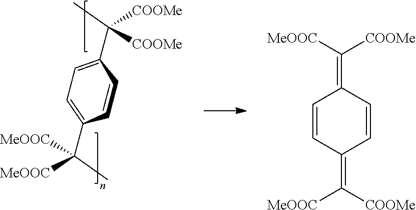

polyQDM-Me (50.0 mg) were mixed with diphenyl ether (5 mL) in a 25 mL round-bottom flask with a condenser under argon atmosphere. The reaction mixture was heated at reflux (˜258° C.) for 30 min. The reaction mixture was cooled down and purified by a short silica column (CH₂Cl₂:Ethyl Acetate=10:1) to give 46.4 mg QDM-Me as a light-yellow powder. (92% yield) ¹H NMR (400 MHz, CDCl₃, ppm) δ: 7.43 (s, CH═, 4H), 3.84 (s, OCH₃, 12H).

Note, the depolymerization reaction also occurred at lower temperature in boiling trichlorobenzene (˜214° C.), but with longer reaction time of 1.5 h, affording 61% recovery yield (26.5 mg) of QDM-Me monomer.

### Example 28

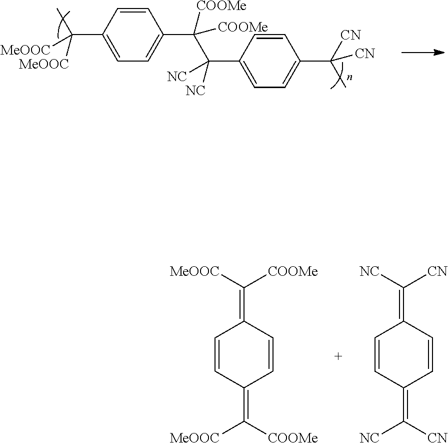

polyQDM-TCNQ (50.0 mg) was mixed with diphenyl ether (5 mL) in a 25 mL round-bottom flask with a condenser under argon atmosphere. The reaction mixture was heated at reflux (−258° C.) for 2 hours. The reaction mixture was cooled down and purified by a silica column (CH₂Cl₂:Ethyl Acetate=10:1) to give 14.1 mg QDM-Me as a light-yellow powder, and 7.0 mg TCNQ as a yellow powder, which gradually changed to yellow-green color over time in air. (45% yield, based on QDM-Me) QDM-Me: ¹H NMR (400 MHz, CDCl₃, ppm) δ: 7.43 (s, CH═, 4H), 3.84 (s, OCH₃, 12H). TCNQ: ¹H NMR (400 MHz, CDCl₃, ppm) δ: 7.56 (s, CH═, 4H).

### Example 29

Grinding depolymerization of polyBIT-8: 40 mg of fresh polyBIT-8 were put in a standard form agate mortar and pestle with O.D. 95 mm. The polymer was continuously ground for 5 minutes and then washed with CH₂C₂. The remaining polymer was filtered and ground again. The grinding process was repeated five times, and the filtrate was dried with reduced pressure to obtain BIT-8 monomer (24 mg recycled, 60% yield).

Examples of film fabrication of polymer crystals via ultra-sonication processing.

### Example 30

PolyME-OMeB film: polyME-OMeB crystals (200 mg) were mixed with acetone (35 mL) in a 50 mL beaker. The ultra-sonication probe was then immersed into the solvent. Ultra-sonication parameters were set as follows: amplitude 20, 4 sec pulse and 1 sec rest. The mixture was cooled by an ice-salt bath during the entire 6 h ultra-sonication processing. The resulting white suspension was divided equally into four portions. Filtration of each portion with a vacuum filtration apparatus and a nylon membrane filter provided a free-standing, flexible white polymer film (around 50 mg each), with quantitative overall yield.

### Example 31

PolyME-CB film: polyME-CB crystals (200 mg) were mixed with chloroform (35 mL) in a 50 mL beaker. The ultra-sonication probe was then immersed into the solvent. Ultra-sonication parameters were set as follows: amplitude 20, 4 sec pulse and 1 sec rest. The mixture was cooled by an ice-salt bath during the entire 4 h ultra-sonication processing. The resulting off-white suspension was divided equally into four portions. Filtration of each portion with a vacuum filtration apparatus and a nylon membrane filter provided a free-standing, flexible white polymer film (around 50 mg each), with quantitative overall yield.

### Example 32

PolyBIT-8 film: polyBIT-8 crystals (200 mg) were mixed with chloroform (35 mL) in a 50 mL beaker. The ultra-sonication probe was then immersed into the solvent. Ultra-sonication parameters were set as follows: amplitude 15, 4 sec pulse and 1 sec rest. The mixture was cooled by an ice-salt bath during the entire 1 h ultra-sonication processing. Longer ultra-sonication time would lead to a more brittle film. The resulting pale-yellow suspension was divided equally into four portions. Filtration of each portion with a vacuum filtration apparatus and a nylon membrane filter provided a free-standing, flexible, pale-yellow polymer film (around 50 mg each), with quantitative overall yield.

Examples of Solution Processing of polyME-OMeB Crystals

### Example 33

polyME-OMeB crystals (3.0 g) were mixed with diphenyl ether (3 mL) in a 25 mL round-bottom flask under argon atmosphere. The reaction mixture was heated at 185° C. for 15 min. The resulting viscous polymer solution was cooled down and poured into 100 mL methanol. The sticky polymer residue was left at the bottom of the flask. This polymer (with solvent residues) was dried in a vacuum oven at 80° C. for 24 hours to obtain a hard white polymer solid, which is amorphous measured by powder XRD. (2.8 g, 93%)

Examples of Heat-Pressing of the Polymer Thin Films.

### Example 34

PolyME-OMeB sonicated-processed film: One piece of polyME-OMeB pristine film was placed between two pieces of smooth aluminum foil (glossy sides facing the film). This sandwich structure was pressed under around 5 MPa pressure at room temperature for 2.5 min, then heat-pressed under around 5 MPa pressure at 120° C. for 5 min. The aluminum foils were carefully removed, and a semi-transparent and glossy polymer thin film was obtained.

### Example 35

PolyME-OMeB solution-processed film: 100 mg of polyME-OMeB solution-processed solid were placed between two pieces of weighing paper. This sandwich structure was pressed under around 1 MPa pressure at room temperature for 2.5 min, then heat-pressed under around 1 MPa pressure at 70° C. for 2.5 min. The weighing papers were carefully removed, and a semi-transparent polymer thin film was obtained.

### Example 36

PolyME-CB film: One piece of polyME-CB pristine film was placed between two pieces of smooth aluminum foil. This sandwich structure was pressed under around 5 MPa pressure at room temperature for 2.5 min, then heat-pressed under around 5 MPa pressure at 120° C. for 5 min. The aluminum foils were carefully removed, and a semi-transparent and glossy polymer thin film was obtained.

### Example 37

PolyBIT-8 film: One piece of polyBIT-8 pristine film was placed between two pieces of smooth aluminum foil. This sandwich structure was heat-pressed under around 5 MPa pressure at 50° C. for 2.5 min. The aluminum foils were carefully removed (if they were stuck with the polymer film, acetone could be applied to wet the interface). The resulting polymer thin film was immersed in acetone for 30 seconds to remove trace amount of BIT-8 monomer produced from mechano-depolymerization, and a glossy pale-yellow polymer-thin film was obtained.

Examples of Preparation of the Polymer Samples for Tensile Stress-Strain Tests.

### Example 38

This method applies for both solution-processed films and sonication-processed films. Roughly 25 mm×5 mm strips were cut from heat-pressed polymer thin films for tensile stress-strain tests. Both ends of the polymer strip were bonded to a piece of polyethylene terephthalate (PET) sheet using epoxy glue to form a “dog-bone” shaped sample. The initial length for (engineering) strain calculation was estimated by the distance between two PET sheets, while the cross-sectional area was estimated by width multiplied by thickness of the strip. For testing, both edges of PET sheets should be aligned with edges of the fixtures or be exposed to ensure a legitimate initial length.

Example of Extrusion and 3D Printing of Solution-Processed polyME-OMeB

### Example 39

polyME-OMeB solid (5.0 g) was fed into a micro-compounder, which was set at 180° C. The polymer was molten and stable at a setting of 170° C. and 2000 rpm in the micro-compounder. The off-white polymer filament with a diameter of roughly 1.6 mm was extruded and collected. The extruded polymer filament was then fed into the 3D printer to print the “P” shape object. 3D-printing parameters are shown in the section below.

Example of Stress-Induced Depolymerization of polyBIT-8 Film.

### Example 40

PolyBIT-8 film was folded twice (four layers, around 0.4 mm thick) and placed between two pieces of weighing paper. A pressure of approximately 1 GPa was applied to the polymer at room temperature for 7.5 min (assuming that the force was only applied to the thick polymer film area). The color of polyBIT-8 film surface changed from pale yellow to dark. This dark substance was washed off the film with deuterated chloroform to form an orange solution. ¹H-NMR characterization showed that the solute is BIT-8 monomer.

Example of Separation of polyBIT-8 and polyME-CB from a Mixture of Common Polymers.

### Example 41

polyBIT-8 (100.1 mg), polyME-CB (100.5 mg), low-density polyethylene (50.2 mg, Mn=7700, PDI=4.5, Aldrich), polypropylene (51.7 mg, amorphous, Aldrich), polystyrene (61.0 mg, Mw=192000, Aldrich), polycarbonate (48.9 mg, T_{g}=200° C., PolyK Technologies), and polyetherimide (55.1 mg, T_{g}=260° C., PolyK Technologies) were mixed with diphenyl ether (6 mL) in a 25 mL round-bottom flask with a condenser under argon atmosphere. The reaction mixture was heated at 160° C. for 30 min. The reaction setup was cooled down and poured into 100 mL hexanes. Filtration of this mixture gave a filtrate containing the crude BIT-8 monomer and polymer precipitate. Diphenyl ether was removed from the BIT-8 filtrate by a short silica column (CH₂Cl₂ as eluent). 100 mg of BIT-8 were recovered (>99% yield). The polymer precipitate containing polyME-CB, LDPE, PP, PS, PC and PEI was mixed with diphenyl ether (6 mL) in a 25 mL round-bottom flask with a condenser under argon atmosphere. The reaction mixture was heated at reflux (˜258° C.) for 20 min. The reaction setup was cooled down quickly by water (to prevent from possible thermal polymerization-depolymerization equilibrium) and poured into 50 mL hexanes. Filtration of this mixture gave a filtrate containing crude ME-CB monomers and polymer precipitate. Diphenyl ether was removed from the ME-CB filtrate by a short silica column (CH₂Cl₂:Ethyl Acetate=15:1). 84.0 mg of ME-CB monomers (with EE, EZ and ZZ isomers) were recovered (84% yield).

What is claimed is:

1. A crystalline polymer comprising:
at least two monomers, independently selected from the group consisting of Formula I, Formula II, and Formula III,
wherein Formula I is 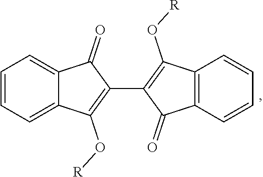
wherein each R is independently a C₁ to C₂₀ linear chain alkyl or a C₁ to C₂₀ branched chain alkyl, in which any —H may be substituted with —F,
wherein Formula II is 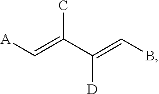
wherein A, B, C and D are independently selected from —C(═O)—R, —C(═O)—OR, 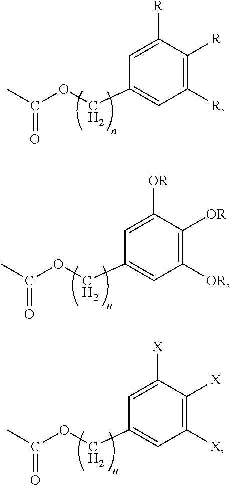
—C(═S)—R, 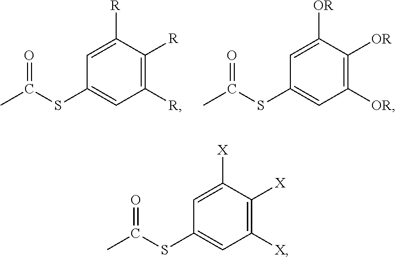
—C(═O)—NRR, 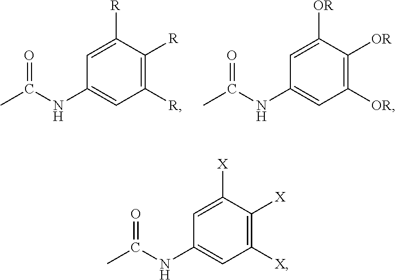
—C(═O)OM, —S(═O)OM, —S(═O)(═O)OM, 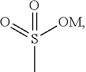
—NO₂, —OR, —OC(═O)R, 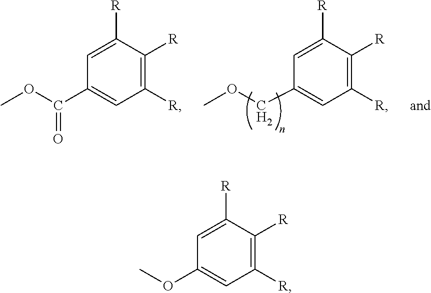
—H, —F, —Cl, and —CH₃,
wherein each R is independently H, —NO₂, a C₁ to C₂₀ branched chain alkyl, or a C₁ to C₂₀ straight chain alkyl, wherein the branched chain alkyl or the straight chain alkyl is optionally substituted with —O—, —NRR or —S—, and any hydrogen can be substituted with —F,
wherein X is a halogen or hydrogen, at least one X is a halogen, M is an organic or inorganic cation, and n=0 to 20 and
wherein Formula III is 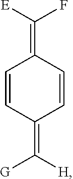
wherein each E, F, G, and H is independently —C(C═O)OR or CN, wherein each R is independently a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein the monomers are chemically bound to each other by at least one carbon-carbon single bond to form the crystalline polymer.

2. The crystalline polymer of claim 1, wherein the carbon-carbon bond has a bond length between about 1.57 Å and about 1.70 Å.

3. The crystalline polymer of claim 2, wherein the polymer has a depolymerization temperature between about 125° C. to about 375° C.

4. The crystalline polymer of claim 1, wherein at least one of A and B is

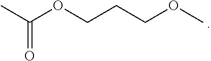

5. The crystalline polymer of claim 1, wherein the organic cation is 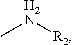
wherein R₂ is H, —CH₂-naphtyl, —CH₂-phenyl, methyl, ethyl, n-propyl, iso-propyl, butyl, n-butyl, sec-butyl, or iso-butyl.

6. A method of topochemically producing a polymer, comprising:
providing a crystalline polymer formed from at least two monomers independently selected from the group consisting of Formula I, Formula II, and Formula III,
wherein Formula I is 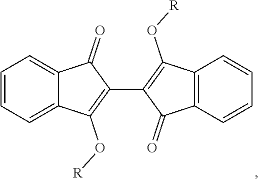
wherein each R is independently a C₁ to C₂₀ linear chain alkyl or a C₁ to C₂₀ branched chain alkyl, wherein any —H maybe substituted with —F,
wherein Formula II is 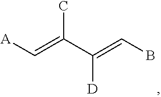
wherein A, B, C and D are independently selected from —C(═O)—R, —C(═O)—OR, 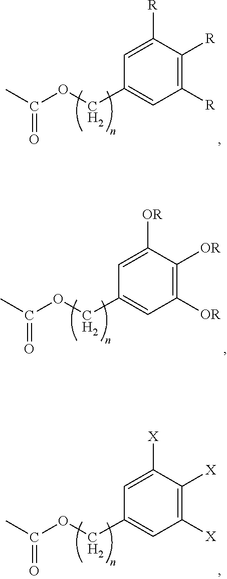
—C(═S)—R, 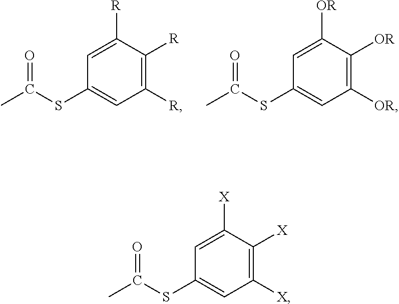
—C(═O)—NRR, 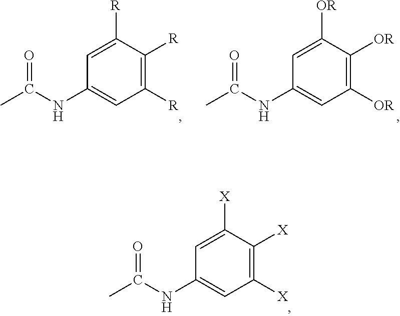
—C(═O)OM, —S(═O)OM, —S(═O)(═O)OM, 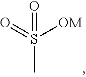
—NO₂, —OR, —OC(═O)R, 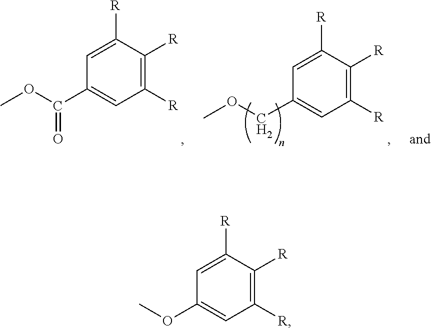
—H, —F, —Cl, and —CH₃,
wherein each R is independently H, —NO₂, a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein X is a halogen or hydrogen, at least one X is a halogen, M is an organic or inorganic cation, and n=0 to 20 and
wherein Formula III is 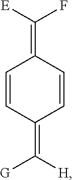
wherein each E, F, G, and H is independently —C(C═O)OR or —CN, wherein each R is independently a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein the monomers are chemically bound to each other by at least one carbon-carbon single bond to form the crystalline polymer;
mixing the crystalline polymer with a solvent to form a mixture;
sonicating the mixture to form a suspension; and
vacuum filtering and heat pressing the suspension.

7. The method of claim 6, wherein the carbon-carbon single bond has a bond length between about 1.57 Å and about 1.70 Å.

8. The method of claim 7, wherein the polymer has a depolymerization temperature between about 125° C. to about 375° C.

9. The method of claim 6, wherein the solvent is an organic solvent.

10. The method of claim 9, wherein the solvent is acetone.

11. The method of claim 9, wherein the solvent is chloroform.

12. The method of claim 6, wherein the ultrasound is performed at an amplitude of about 20, about 4 second pulse and about 1 second rest.

13. The method of claim 12, wherein the ultrasound is performed at an amplitude of about 15, about 4 second pulse and about 1 second rest.

14. The method of claim 14, wherein the ultrasound is performed at a temperature below 0° C.

15. The method of claim 6, wherein A and B are

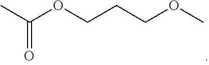

16. The method of claim 6, wherein the organic cation is 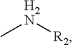
wherein R₂ is H, —CH₂-naphtyl, —CH₂-phenyl, methyl, ethyl, n-propyl, iso-propyl, butyl, n-butyl, sec-butyl, or iso-butyl.

17. A method of thermoplastic production of a polymer, comprising:
providing a crystalline polymer formed from at least two monomers independently selected from the group consisting of Formula I, Formula II, and Formula III,
wherein Formula I is 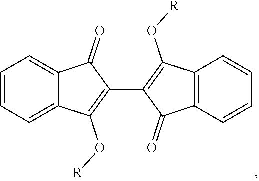
wherein each R is independently C₁ to C₂₀ linear chain alkyl or a C₁ to C₂₀ branched chain alkyl, wherein any —H may be substituted with —F,
wherein Formula II is 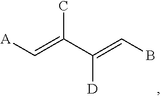
wherein A, B, C and D are independently selected from —C(═O)—R, —C(═O)—OR, 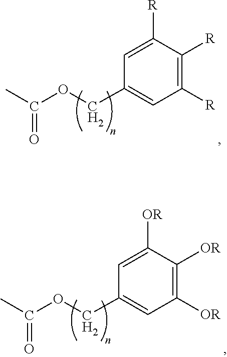
—C(═S)—R, 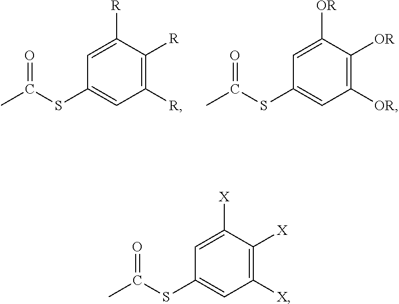
—C(═O)—NRR, 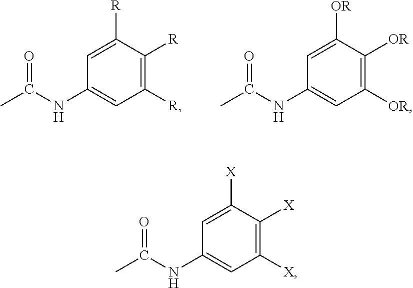
—C(═O)OM, —S(═O)OM, —S(═O)(═O)OM, 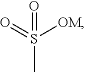
—NO₂, —OR, —OC(═O)R, 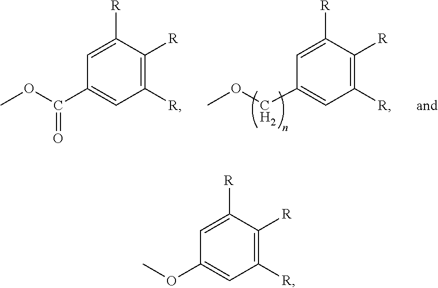
—H, —F, —Cl, and —CH₃,
wherein each R is independently H, —NO₂, a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein X is a halogen or hydrogen, at least one X is a halogen, M is an organic or inorganic cation, and n=0 to 20, and
wherein Formula III is 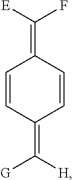
wherein each E, F, G, and H is independently —C(C═O)OR or —CN, wherein each R is independently a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein the monomers are chemically bound to each other by at least one carbon-carbon single bond to form the crystalline polymer;
mixing the crystalline polymer with a first solvent to form a mixture;
subjecting the mixture to heat to form a viscous polymer solution;
mixing the viscous polymer solution with a second solvent to form a residue; and
drying the residue.

18. The method of claim 17, wherein the carbon-carbon single bond has a bond length between about 1.57 Å and about 1.70 Å.

19. The method of claim 18, wherein the polymer has a depolymerization temperature between about 125° C. to about 375° C.

20. The method of claim 17, wherein A and B are

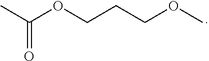

21. A method of depolymerization, comprising:
providing a crystalline polymer formed from at least two monomers independently selected from the group consisting of Formula I, Formula II, and Formula III,
wherein Formula I is 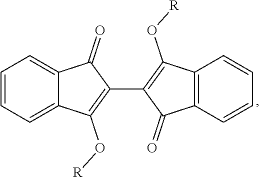
wherein each R is independently a C₁ to C₂₀ linear chain alkyl or a C₁ to C₂₀ branched chain alkyl, wherein any —H maybe substituted with —F,
wherein Formula II is 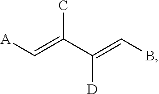
wherein A, B, C and D are independently selected from —C(═O)—R, —C(═O)—OR, 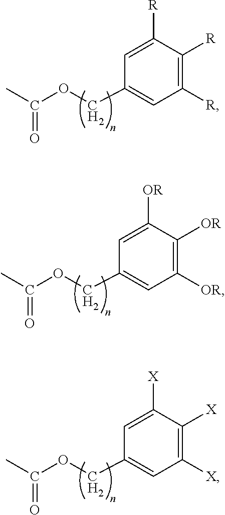
—C(═S)—R, 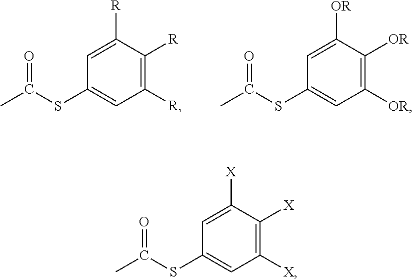
—C(═O)—NRR, 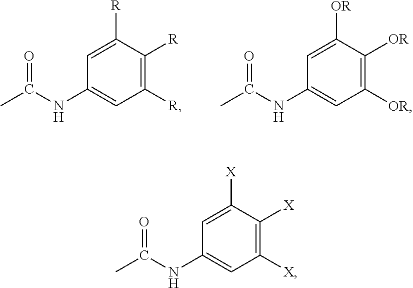
—C(═O)OM, —S(═O)OM, —S(═O)(═O)OM, 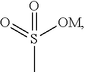
—NO₂, —OR, —OC(═O)R, 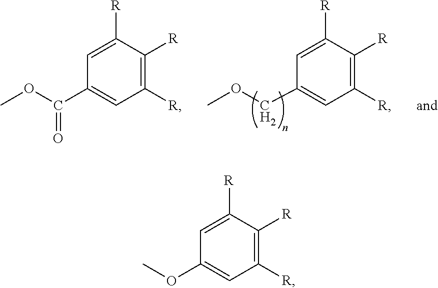
—H, —F, —Cl, and —CH₃,
wherein each R is independently H, —NO₂, a C₁ to C₂₀ branched chain alkyl or a C₁ to C₂₀ straight chain alkyl, wherein the branched chain alkyl or the straight chain alkyl is optionally substituted with —O—, —NRR or —S—, and any hydrogen can be substituted with —F,
wherein X is a halogen or hydrogen, at least one X is a halogen, M is an organic or inorganic cation, and n=0 to 20, and
wherein Formula III is 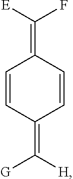
wherein each E, F, G, and H is independently —C(C═O)OR or —CN, wherein each R is independently a C₁ to C₂₀ branched chain alkyl or a C₁ to C₂₀ straight chain alkyl, optionally substituted with —O—, —NRR or —S—, and any hydrogen can be substituted with —F,
wherein the monomers are chemically bound to each other by at least one carbon-carbon single bond to form the crystalline polymer,
wherein the carbon-carbon single bond has a bond length between about 1.57 Å and about 1.70 Å,
mixing the crystalline polymer with a solvent to form a mixture,
heating the mixture to a temperature between about 125° C. to about 375° C., and
recovering the monomers.

22. The method of claim 21, wherein the temperature is between about 240° C. and about 275° C. or between about 310° C. and about 355° C.

23. The depolymerization process of claim 22, wherein the carbon-carbon single bond has a bond length between about 1.57 Å and about 1.70 Å.

24. The method of claim 21, wherein A and B are

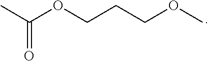

25. The depolymerization process of claim 21, wherein the organic cation is 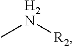
wherein R₂ is H, —CH₂-naphtyl, —CH₂-phenyl, methyl, ethyl, n-propyl, iso-propyl, butyl, n-butyl, sec-butyl, or iso-butyl.
